(19) United States
(12) Patent Application Publication
(10) Pub. No.: US 2026/0193229 A1
(43) Pub. Date: Jul. 9, 2026

(54) BRISC DEUBIQUITINATING ENZYME INHIBITORS

(71) Applicants: THE TRUSTEES OF THE UNIVERSITY OF PENNSYLVANIA, Philadelphia, PA (US); SINAI HEALTH SYSTEM, Toronto (CA); DREXEL UNIVERSITY, Philadelphia, PA (US); THE WISTAR INSTITUTE, Philadelphia, PA (US); UNIVERSITY OF LEEDS, Leeds (GB)

(72) Inventors: Roger GREENBERG, Wynnewood, PA (US); Frank SICHERI, Toronto (CA); Elton ZEQIRAJ, Leeds (GB); Joseph M. SALVINO, Chester Springs, PA (US)

(21) Appl. No.: 19/134,401

(22) PCT Filed: Nov. 30, 2023
(86) PCT No.: PCT/EP2023/083851

§ 371 Date: May 30, 2025

Related U.S. Application Data
(60) Provisional application No. 63/385,576, filed on Nov. 30, 2022.

(51) Int. Cl. C07D 401/12; A61K 31/454; A61P 37/06

(52) U.S. Cl. CPC C07D 401/12; A61K 31/454; A61P 37/06

(57) ABSTRACT

The present disclosure provides for techniques using BRISC deubiquitinating enzyme inhibitors of formula (I) for treating and/or preventing systemic lupus erythematosus (SLE). Accordingly, the present disclosure provides for compositions and methods for treating or preventing a disease or disorder mediated by inhibition of type I interferon receptor (IFNAR1) signaling.

### STATEMENT OF GOVERNMENT INTEREST

This invention was made with government support under Grant No. CA138835 awarded by the National Institute of Health. The government has certain rights in the invention.

### BACKGROUND

Certain inflammatory disorders, including systemic lupus erythematosus (SLE), septic shock, psoriasis, and type II diabetes can cause the immune system to attack cells or tissue, and can result in abnormal inflammation and swelling, tissue damage, and chronic pain. For example, SLE is a chronic and disabling autoimmune disease in which the human immune system becomes hyperactive and attacks normal healthy tissues. Lupus impacts multiple organ systems such as joints, skin, kidneys, blood cells, heart, and lungs and is strongly associated with elevated inflammatory cytokine activation. It is estimated that 5-10% of the SLE patients develop end stage renal disease within 10 years of diagnosis.

An estimated 5 million people worldwide have some form of lupus disease, and the disease affects an estimated 520,000 patients in the United States. Despite its prevalence, there remains a large and unmet need for effective new therapies beyond Benlysta.

It is known that inhibition of type I interferon receptor (IFNAR1) signaling mitigates lupus pathogenesis in mouse models and in human patients. Thus, there is a need to develop new agents effective against lupus and other inflammatory disorders, including agents that reduce interferon dependent activity.

### SUMMARY

In certain embodiments, the disclosure relates to a compound of Formula I.

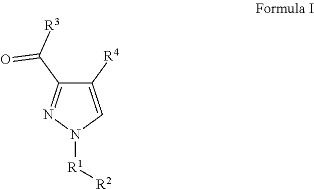

or a salt, ester, or solvate thereof, wherein R¹ is C═O, SO₂-Me, carbonyl, or a sulfonyl; wherein R² is optional, and if present, is CF₃, C₁₋₆alkyl, substituted C₁₋₆alkyl, amino, alkoxycarbonyl, aryl, substituted aryl, benzyl, or a substituted naphthalene; wherein R³ is a substituted amino; and wherein R⁴ is H or an amino.

In certain embodiments, the disclosure relates to a compound of Formula II:

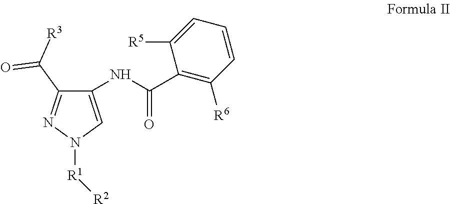

or a salt, ester, or solvate thereof, wherein R¹ is C═O, SO₂-Me, amide, carbonyl, or a sulfonyl; wherein R² is optional, and if present, is CF₃, C₁₋₆alkyl, substituted C₁₋₆alkyl, amino, alkoxycarbonyl, aryl, substituted aryl, benzyl, or a substituted naphthalene; wherein R³ is a substituted amino; and wherein R⁵ and R⁶ are independently H or a halo.

In certain embodiments, the disclosure relates to a compound of Formula II:

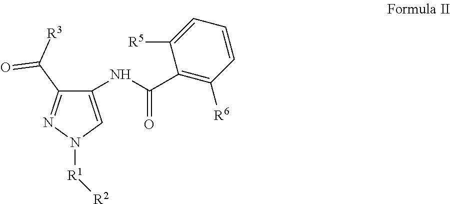

or a salt, ester, or solvate thereof, wherein R¹ is C═O, SO₂-Me, amide, carbonyl, or a sulfonyl; wherein R² is optional, and if present, is CF₃, C₁₋₆alkyl, substituted C₁₋₆alkyl, amino, alkoxycarbonyl, aryl, substituted aryl, benzyl, or a substituted naphthalene; wherein R³ is a substituted or unsubstituted amino; and wherein R⁵ and R⁶ are independently H or a halo.

In certain embodiments, the disclosure relates to a compound of Formula III:

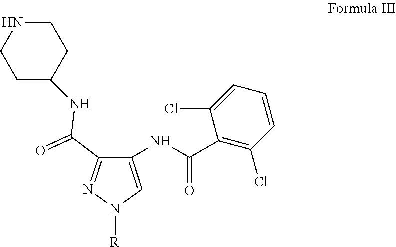

or a salt, ester, or solvate thereof, wherein R is H, SO₂-Me, benzoyl, substituted benzoyl, or a sulfonyl.

In certain embodiments R is SO₂-Me or CO-2,6-dichlorobenzoyl.

In certain embodiments, the present disclosure provides methods of using the compounds of Formulas I-III for inhibiting BRISC holo-enzymes, comprised of BRCC36, KIAA0157, BRCC45, and MERIT40. In certain embodiments, the present disclosure provides methods of using the compounds for inhibiting BRISC enzymes in a subject, e.g., a human subject, including administering to the subject a therapeutically effective amount of a compound of Formulas I-III.

In certain embodiments, the present disclosure provides methods of using the compounds of Formulas I-III for inhibiting type I interferon receptor (IFNAR1) signaling. In certain embodiments, the present disclosure provides methods of using the compounds for inhibiting BRISC enzymes in a subject, e.g., a human subject, including administering to the subject a therapeutically effective amount of a compound of Formulas I-III.

In certain embodiments, the present disclosure provides methods of using the compounds of Formulas I-III for treating or preventing an autoimmune disease in a subject, e.g., a human subject, including administering to the subject a therapeutically effective amount of a compound of Formulas I-III.

In certain embodiments, the present disclosure provides methods of using the compounds of Formulas I-III for treating or preventing an inflammatory disease in a subject, e.g., a human subject, including administering to the subject a therapeutically effective amount of a compound of Formulas I-III.

In certain embodiments, the present disclosure provides methods of using the compounds of Formulas I-III for treating or preventing systemic lupus erythematosus (SLE) in a subject, e.g., a human subject, including administering to the subject a therapeutically effective amount of a compound of Formulas I-III.

certain embodiments, the present disclosure provides methods of using the compounds of Formulas I-III for treating or preventing systemic lupus erythematosus (SLE), systemic sclerosis, scleroderma, myositis, or Sjögren's syndrome in a subject, e.g., a human subject, including administering to the subject a therapeutically effective amount of a compound of Formulas I-III.

The compounds of Formula I-III or any other compounds contemplated by the present invention may be for use in any of the above methods of treatment or methods of use or any other method disclosed herein.

In certain embodiments, the BRISC inhibitor is administered in combination with a second therapeutic agent.

Another aspect of the disclosure provides a pharmaceutical composition comprising at least one compound according to Formulas I-III and a pharmaceutically acceptable carrier. In certain embodiments, the pharmaceutical composition comprises at least one of a pharmaceutically acceptable diluent, carrier, solubilizer, emulsifier, preservative, and adjuvant.

The present disclosure also provides kits comprising a container for the compound or pharmaceutical composition, and instructions for use.

### BRIEF DESCRIPTION OF THE DRAWINGS

FIG. 1. Schematic of K63-Ub dependent networks are activated in response to cytokines. BRISC DUB activity stabilizes IFNAR1 and can affect TLR mediators to promote type I interferon signals.

FIG. 2A-FIG. 2F. BRISC targets IFNAR1. (FIG. 2A) IFN (interferon) treatment increases IFNAR1 interaction with SHMT2. (FIG. 2B) Kiaa0157−/− cells have elevated IFNAR1 K63-ubiquitination. (FIG. 2C-FIG. 2D) Kiaa0157 null MEFs and mice show reduced interferon signaling in response to LPS. (FIG. 2E-FIG. 2F) Kiaa0157−/− mice have reduced lung injury and mortality after LPS administration.

FIG. 3A-FIG. 3C. BRISC null mice are resistant to pristane induced SLE. (FIG. 3A) Reduced peritoneal cells in Kiaa0157^{-/-} mice at 2 weeks after IP (TMPD). (FIG. 3B, FIG. 3C) Kiaa0157^{-/-} mice show reduced anti-nuclear antibodies compared to +/+ controls at 6 months after IP TMPD.

FIG. 4A-FIG. 4B. BRISC is not epistatic to IFNAR1. (FIG. 4A, FIG. 4B) double knockout cells have reduced peritoneal cells and CXCL10 at 2 weeks post TMPD compared to single knockouts.

FIG. 5. KIAA0157−/− RAW264.7 macrophages show reduced IFNβ production and ISG15 during TLR7 activation.

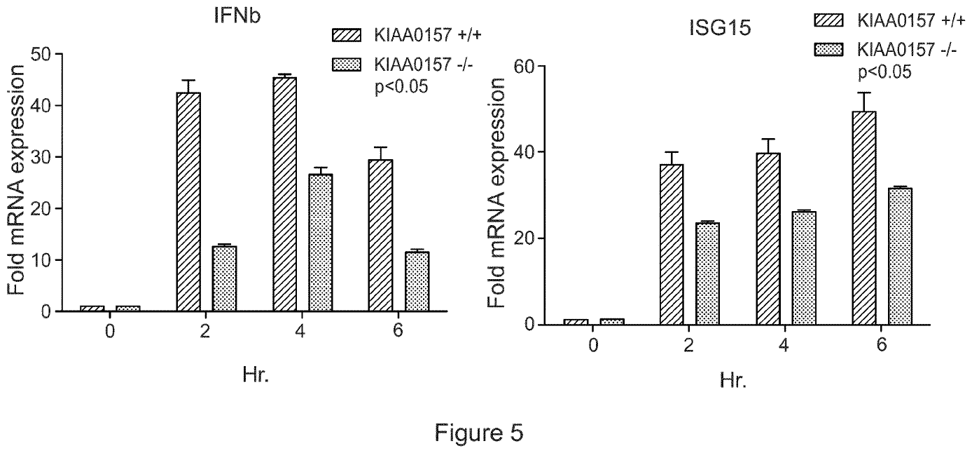

FIG. 6A-FIG. 6C. Development of BRISC DUB Inhibitor High Throughput Screening. (FIG. 6A) Baculoviral expression of BRISC holo-enzyme. (FIG. 6B) Quenched K63-linked-di-Ub substrate. (FIG. 6C) Progress curves of BRISC enzyme activity showing cleavage of K63 di-Ubiquitin (di-Ub). Reaction was monitored over time by reading fluorescence of TAMRA labeled Ub (Ub-TM), which is liberated after DUB cleavage of the quenched K63-linked di-Ub substrate.

FIG. 7A-FIG. 7B. Development of first-in-class BRISC DUB Inhibitors. (FIG. 7A) The lead compound specific BRISC inhibitor show low micromolar inhibition of BRISC but not the unrelated USP2 and trypsin enzymes. The lead compound is also specific for inhibition of BRISC, and does not cross-react with the related ARISC (BRCC36-Abraxas-BRCC45-MERIT40) and RAP80 (BRCC36-Abraxas-BRCC45-MERIT40-RAP80) complexes. (FIG. 7B) Structure of BRISC inhibitors (top). Compound 2 inhibits interferon signaling in response to infection with HSV lacking lytic phase gene ICPO (bottom). The right side of the blot is from KIAA0157 null cells and shows reduced Stat1 phosphorylation independent of the inhibitor Compound 2.

### DETAILED DESCRIPTION

Systemic lupus erythematosus (SLE) is a chronic autoimmune disease that affects an estimated 5 million people worldwide, including about 520,000 patients in the United States as of the filing of the present disclosure. Despite its prevalence, there remains a large and unmet need for effective new therapies.

Lupus impacts multiple organ systems and is strongly associated with elevated inflammatory cytokine activation. Indeed, inhibition of type I interferon receptor (IFNAR1) signaling mitigates lupus pathogenesis in mouse models and in human patients. There are no approved means to inhibit Type I interferon responses. Agents that reduce interferon dependent gene expression represent novel and effective strategies against lupus.

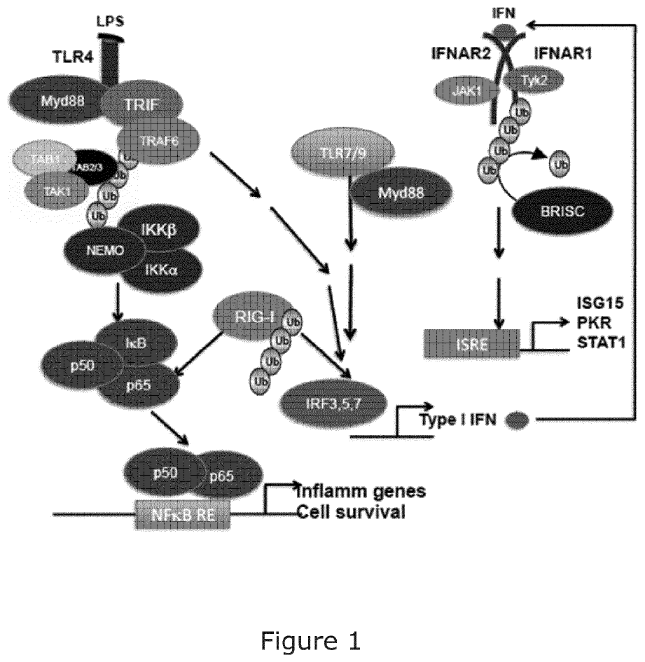

Ubiquitin is a versatile post-translational modification that can form chains through different linkages comprised of an epsilon amino group from one of 7 different lysines on one ubiquitin monomer and the C-terminal glycine of another ubiquitin molecule. Lysine63-linked ubiquitin chains (K63-Ub) are vital to inflammatory cytokine signaling (FIG. 1).

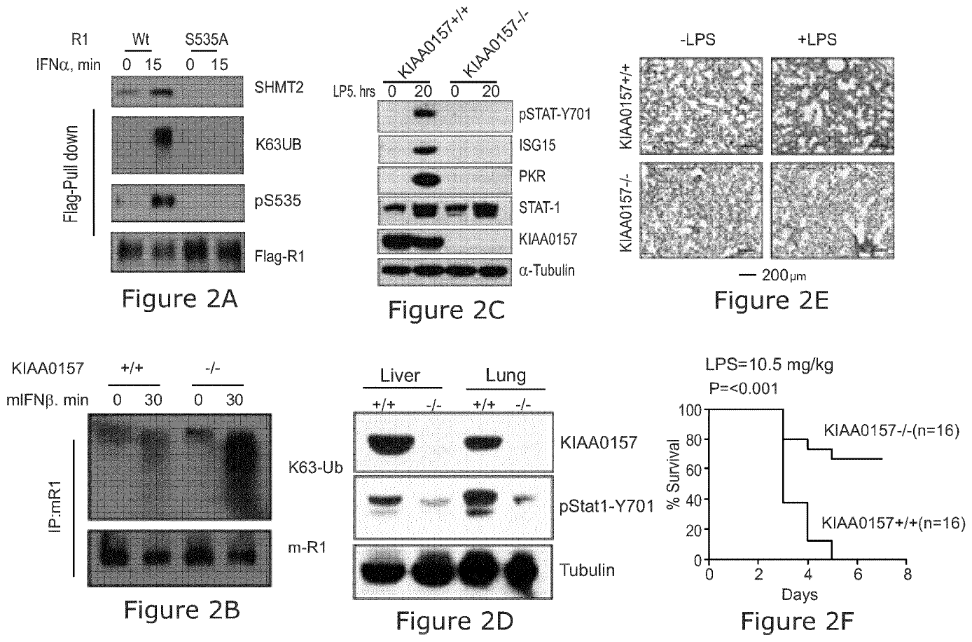

The BRISC complex is composed of four core constituents (KIAA0157, MERIT40, BRCC45, and BRCC36) and is the major K63-Ub specific DUB activity present in the cytoplasm. BRCC36, the enzymatic subunit, belongs to a small class of Zn2+-dependent ubiquitin proteases (JAMM domain DUBs) that are commonly found in association with large protein complexes. As demonstrated below, BRISC deubiquitinates actively engaged Type 1 interferon receptor (IFNAR1) in both human and mouse cells to promote signaling downstream of interferon-α and -β (FIGS. 2A, B). BRISC is required for maximal type I interferon receptor signaling.

IFNAR1 becomes hyper K63-ubiquitinated in cells that have a targeted deletion of the BRISC specific subunit, Kiaa0157, which is required for BRISC stability and DUB activity. Kiaa0157^{-/-} cells show more rapid IFNAR1 internalization and degradation in the lysosome following interferon treatment. As a consequence, BRISC deficiency mitigates interferon signaling, tissue damage, and mortality in mice exposed to lipopolysaccharide (LPS) (FIG. 2), indicating that BRISC DUB inhibition a novel target for diseases arising from elevated interferon signaling.

As demonstrated in the examples below, data shows that BRISC deubiquitinating enzyme inhibitors are useful in conditions stemming from elevated interferon responses (e.g., SLE (IFN pathway), Scleroderma (TLR4 Signaling)). As further demonstrated in the examples below, first-in-class BRISC inhibitors have been identified, and crystallization of BRISC sub-complex in active (e.g., BRCC36-KIAA0157) and inactive (e.g., BRCC36) conformations has been completed to facilitate structure-guided drug design.

The term “BRISC deubiquitinating enzyme inhibitor” or “BRISC inhibitor” as used herein means a molecule having the ability to inhibit a biological function of a native BRISC deubiquitinating enzyme. Accordingly, the term “inhibitor” is defined in the context of the biological role of BRISC. While preferred inhibitors herein specifically interact with (e.g., bind to) a BRISC, molecules that inhibit a BRISC biological activity by interacting with other members of signal transduction pathways involving BRISC are also specifically included within this definition. In certain embodiments, a BRISC biological activity inhibited by a BRISC inhibitor is associated with the inhibition of Type 1 interferon receptor (IFNAR1) in addition to toll like receptors 4 and 7 (TLR4 and TLR 7). In certain embodiments, a BRISC biological activity inhibited by a BRISC inhibitor is associated with treatment or prevention of SLE or scleroderma. BRISC inhibitors can also have utility in septic shock and in other conditions emanating from chronic inflammatory signaling such as psoriasis and type II diabetes.

The definition of each expression, e.g., alkyl, m, n, and the like, when it occurs more than once in any structure, is intended to be independent of its definition elsewhere in the same structure.

As used herein, “substitution” or “substituted with” includes the implicit proviso that such substitution is in accordance with permitted valence of the substituted atom and the substituent, and that the substitution results in a stable compound, e.g., a compound which does not spontaneously undergo transformation such as by rearrangement, cyclization, elimination, or other reaction.

The term “substituted” is also contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein below. The permissible substituents can be one or more and the same or different for appropriate organic compounds. For purposes herein, the heteroatoms such as nitrogen can have hydrogen substituents, and/or any permissible substituents of organic compounds described herein which satisfy the valences of the heteroatoms. This disclosure is not intended to be limited in any manner by the permissible substituents of organic compounds.

The term “saturated,” as used herein, pertains to compounds and/or groups which do not have any carbon-carbon double bonds or carbon-carbon triple bonds.

The term “unsaturated,” as used herein, pertains to compounds and/or groups which have at least one carbon-carbon double bond or carbon-carbon triple bond.

The term “aliphatic,” as used herein, pertains to compounds and/or groups which are linear or branched, but not cyclic (also known as “acyclic” or “open-chain” groups). The term “cyclic,” as used herein, pertains to compounds and/or groups which have one ring, or two or more rings (e.g., spiro, fused, bridged).

The term “aromatic” refers to a planar or polycyclic structure characterized by a cyclically conjugated molecular moiety containing 4n+2 electrons, wherein n is the absolute value of an integer. Aromatic molecules containing fused, or joined, rings also are referred to as bicyclic aromatic rings. For example, bicyclic aromatic rings containing heteroatoms in a hydrocarbon ring structure are referred to as bicyclic heteroaryl rings.

The term “hydrocarbon” as used herein refers to an organic compound consisting entirely of hydrogen and carbon.

The term “heteroatom” as used herein is art-recognized and refers to an atom of any element other than carbon or hydrogen. Illustrative heteroatoms include boron, nitrogen, oxygen, phosphorus, sulfur, and selenium.

The term “alkyl” means an aliphatic or cyclic hydrocarbon radical containing from 1 to about 12 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 2-methylcyclopentyl, and 1-cyclohexylethyl. Any alkyl described herein can be a substituted alkyl.

As used herein, “C₁₋₆alkyl” refers to straight or branched aliphatic groups having from 1 to 6 carbon atoms. Examples include methyl, ethyl, propyl, isopropyl, butyl, isobutyl, tert-butyl, pentyl, isopentyl, hexyl, and the like. “C₁₋₆alkylene” refers to straight or branched aliphatic groups having from 1 to 6 carbon atoms and having two points of attachment, for example, methylene (—CH2-), ethylene (—CH2-CH2-), propylene (—CH2-CH2-CH2-), and the like.

As used herein, “substituted C₁₋₆alkyl” refers to C₁₋₆alkyl groups as defined herein, substituted with another moiety that is, for example, F, Cl, Br, CF₃, carboxy, alkoxycarbonyl, aryl, or heterocycloalkyl.

As used herein “C₃₋₆cycloalkyl” refers to a cyclic aliphatic group having from 3 to 6 carbon atoms. Examples include cyclopropyl, cyclobutyl, cyclopentyl, and cyclohexyl. “C₃₋₆cycloalkylene” refers to a cyclic aliphatic group having from 3 to 6 carbon atoms and having two points of attachment, for example, cyclopropylene, cyclobutylene, cyclopentylene, and cyclohexylene.

“Alkenyl” refers to a monoradical of a branched or unbranched hydrocarbon chain containing at least one double bond. Alkenyl groups can contain 2-10 carbon atoms, such as 2-6 carbon atoms or 2-4 carbon atoms.

“Alkynyl” refers to a monoradical of a branched or unbranched hydrocarbon chain containing at least one triple bond. Alkynyl groups can contain 2-10 carbon atoms, such as 2-6 carbon atoms or 2-4 carbon atoms. The term “carbocyclyl” or “cycloalkyl” as used herein means monocyclic or multicyclic (e.g., bicyclic, tricyclic, etc.) hydrocarbons containing from 3 to about 12 carbon atoms that are completely saturated or have one or more unsaturated bonds, and for the avoidance of doubt, the degree of unsaturation does not result in an aromatic ring system (e.g., phenyl). Examples of carbocyclyl groups include 1-cyclopropyl, 1-cyclobutyl, 2-cyclopentyl, 1-cyclopentenyl, 3-cyclohexyl, 1-cyclohexenyl and 2-cyclopentenylmethyl.

“Heterocycle” refers to 3-15 membered monocyclic, bicyclic, and tricyclic nonaromatic rings, which can be saturated or unsaturated, can be bridged, spiro, and/or fused, and which contain, in addition to carbon atoms, at least one heteroatom, such as nitrogen, oxygen, sulfur or phosphorus. A heterocycle can contain, in addition to carbon atoms, at least one nitrogen, oxygen, or sulfur. A heterocycle can contain from 3 to about 10 ring atoms, 3 to about 7 ring atoms, 5 to 7 ring atoms, 5 ring atoms, 6 ring atoms, or 7 ring atoms. Unless otherwise indicated, the heterocycle can be C-attached or N-attached where such is possible and results in the creation of a stable structure. Examples include, but are not limited to, tetrahydrofuranyl, pyrrolidinyl, pyrrolinyl, imidazolidinyl, imidazolinyl, azetidinyl, pyrazolidinyl, pyrazolinyl, piperidinyl, piperazinyl, indolinyl, isoindolinyl, morpholinyl, thiomorpholinyl, homomorpholinyl, homopiperidinyl, homopiperazinyl, thiomorpholinyl-5-oxide, thiomorpholinyl-S,S-dioxide, tetrahydropyranyl, piperidinyl, tetrahydrothienyl, homothiomorpholinyl-S,S-dioxide, oxazolidinonyl, dihydropyrazolyl, dihydropyrazinyl, dihydropyridinyl, dihydropyrimidinyl, dihydrofuryl, dihydropyranyl, tetrahydrothienyl-5-oxide, tetrahydrothienyl-S,S-dioxide, homothiomorpholinyl-5-oxide, and quinuclidinyl.

As used herein “heterocycloC₃₋₆alkyl” or “heterocycloalkyl” refers to an aliphatic cyclic moiety that includes from 3-6 carbon atoms, in addition to 1, 2 or 3 heteroatoms that are N, O, or S.

As used herein, “halo” or “halogen” refers to F, Cl, Br, or I.

The term “amino” as used herein refers to —NH2 and substituted derivatives thereof wherein one or both of the hydrogens are independently replaced with substituents selected from the group consisting of alkyl, haloalkyl, fluoroalkyl, alkenyl, alkynyl, carbocyclyl, heterocyclyl, aryl, aralkyl, heteroaryl, heteroaralkyl, alkylcarbonyl, haloalkylcarbonyl, fluoroalkylcarbonyl, alkenylcarbonyl, alkynylcarbonyl, carbocyclylcarbonyl (C(O)carbocyclyl), heterocyclylcarbonyl (C(O)heterocyclyl), arylcarbonyl (CC(O)aryl), aralkylcarbonyl (C(O)aralkyl), heteroarylcarbonyl (C(O)heteroaryl), heteroaralkylcarbonyl (C(O)heteroaralkyl) and the sulfonyl and sulfinyl groups defined above; or when both hydrogens together are replaced with an alkylene group (to form a ring which contains the nitrogen). Representative examples include, but are not limited to methylamino, acetylamino, dimethylamino, and dichlorobenzoyl.

“Aryl” refers to 6-15 membered monoradical bicyclic or tricyclic hydrocarbon ring systems, including bridged, spiro, and/or fused ring systems, in which at least one of the rings is aromatic. An aryl group can contain 6 (i.e., phenyl) or about 9 to about 15 ring atoms, such as 6 (i.e., phenyl) or about 9 to about 11 ring atoms. In certain embodiments, aryl groups include, but are not limited to, naphthyl, indanyl, indenyl, anthryl, phenanthryl, fluorenyl, 1,2,3,4-tetrahydronaphthalenyl, 6,7,8,9-tetrahydro-5H-benzocycloheptenyl, and 6,7,8,9-tetrahydro-5H-benzocycloheptenyl. “Heteroaryl” refers to (a) 5 and 6 membered monocyclic aromatic rings, which contain, in addition to carbon atoms, at least one heteroatom, such as nitrogen, oxygen or sulfur, and (b) 7-15 membered bicyclic and tricyclic rings, which contain, in addition to carbon atoms, at least one heteroatom, such as nitrogen, oxygen or sulfur, and in which at least one ring is aromatic. Heteroaryl groups can be bridged, spiro, and/or fused. In further embodiments, a heteroaryl can contain 5 to about 15 ring atoms. In further embodiments, a heteroaryl can contain 5 to about 10 ring atoms, such as 5, 6, 9, or 10 ring atoms. The heteroaryl can be C-attached or N-attached where such is possible and results in the creation of a stable structure.

The abbreviations Me, Et, Ph, Tf, Nf, Ts, SO₂-Me (or Ms), and OMe, represent methyl, ethyl, phenyl, trifluoromethanesulfonyl, nonafluorobutanesulfonyl, p-toluenesulfonyl, methanesulfonyl, and methoxy, respectively. A more comprehensive list of the abbreviations utilized by organic chemists of ordinary skill in the art appears in the first issue of each volume of the Journal of Organic Chemistry; this list is typically presented in a table entitled Standard List of Abbreviations.

As used herein, “stereoisomers” refers to all enantiomerically/diastereomerically pure and enantiomerically/diastereomerically enriched compounds. Atropisomers, that is, stereoisomers resulting from hindered rotation about single bonds, are also within the scope of the term, “stereoisomers.”

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect can be prophylactic in terms of completely or partially preventing a disease or condition, or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease of disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

As used herein, “KIAA0157” refers to the KIAA0157 gene in humans whereas “Kiaa0157” refers to the corresponding gene in mice.

Various aspects of this disclosure are described in further detail in the following subsections.

### 1. Exemplary BRISC Inhibitor Compounds

In certain embodiments, the disclosure relates to a compound of Formula I:

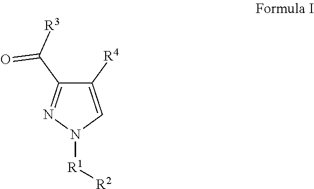

or a salt, ester, or solvate thereof, wherein:
R¹ is C═O, SO₂-Me, carbonyl, or a sulfonyl;
R² is optional, and if present, is CF₃, C₁₋₆alkyl, substituted C₁₋₆alkyl, amino, alkoxycarbonyl, aryl, substituted aryl, benzyl, or a substituted naphthalene, wherein
   the C₁₋₆alkyl is substituted with phenyl substituted with —OC₁₋₆alkyl,
   the aryl group is substituted with one, two or three groups selected from: halo, —OR⁷, haloalkyl, —NH₂, C₁₋₆alkylamino (such as methylamine or dimethylamine), and C₁₋₆alkyl ester;
   the naphthalene is substituted with a C₁₋₆alkylamino;
   the amino is selected from: —NIR;
R³ is a substituted amino, wherein the amino is substituted with two groups (optionally where 1 group is H or Me) selected from: H, C₁₋₆alkyl, —(CH₂)ₘNH₂, substituted or unsubstituted 6 membered heteroaryl, substituted or unsubstituted —(CH₂)ₘ-4 to -6-membered heterocycloalkyl or the two substituents on the amino may form a substituted or unsubstituted 5 or 6 membered ring,
   wherein each group that may be substituted is substituted with one or two C₁₋₆alkyl groups;
R⁴ is H or a substituted amino, wherein the amino group is substituted with benzyl, —C(O)aryl or —C(O)heteroaryl wherein the benzyl group and the aryl group are unsubstituted or substituted with R¹⁰; and
R⁷ is selected from: C₁₋₆alkyl, C₁₋₆alkynyl, —CH₂C(O)OH, —CH₂C(O)O(CH₂)ₙCH₃, —CH₂C(O)O(CH₂)ₙCCH, —CH₂C(O)NH(CH₂)ₙCH₃, —(CH₂)ₙNH₂, —(CH₂)ₙNO₂, and —(CH₂)ₙR⁹, R is phenyl substituted with halo, —CO₂H, —CO₂—C₁₋₆alkyl, and —OC₁₋₆alkyl;
R⁹ is phthalimide;
R¹⁰ is selected from: C₁₋₆alkyl, —OC₁₋₆alkyl, C₁₋₆haloalkyl, halo, —NO₂, —CH₂morpholinyl and diaziridine substituted with C₁₋₆haloalkyl;
n is 0 to 6; and
m is 0 to 4.

In certain embodiments, the aryl is substituted with at least one of a halo group, OMe, CF₃, amino, and COOMe. In certain embodiments, the aryl is substituted with at least one halo group. In certain embodiments, the aryl is substituted with 1-3 halo groups. In certain embodiments, the aryl is substituted with 1 halo group, 2 halo groups, or 3 halo groups. In certain embodiments, the halo group is Cl.

In certain embodiments, the substituted naphthalene is substituted with an amino.

In certain embodiments R² is optional, and if present, is CF₃; C₁₋₆alkyl; amino; aryl; aryl substituted with at least one (optionally one or two) of a halo group, OMe, CF₃, amino, and COOMe; benzyl, or a naphthalene substituted with an amino.

In certain embodiments R² is optional, and if present, is methyl; —NIR; phenyl; phenyl substituted with one or two of a halo group, OMe, CF₃, amino, and COOMe; benzyl, or a naphthalene substituted with a C₁₋₆alkyl amino (such as methylamine or dimethylamine), wherein R⁸ is selected from: halo substituted phenyl, C₁₋₆alkyl ester substituted phenyl, or C₁₋₆alkoxyl substituted phenyl.

In certain embodiments, the amino is substituted with H, SO₂-Me, C₁₋₆alkyl, substituted C₁₋₆alkyl, carbonyl, heterocycle, heteroaryl, benzoyl, substituted benzoyl, substituted benzyl, heterobicycle, or both hydrogens together are replaced with an alkylene group (to form a ring which contains the nitrogen and optionally at least one additional nitrogen). In certain embodiments, the substituted benzoyl is substituted with at least one of a halo group, OMe, CF₃, amino, and COOMe. In certain embodiments, the substituted benzoyl is substituted with at least one halo group. In certain embodiments, the substituted benzoyl is substituted with 1-3 halo groups. In certain embodiments, the substituted benzoyl is substituted with 1 halo group, 2 halo groups, or 3 halo groups. In certain embodiments, the halo group is Cl.

In certain embodiments, the substituted C₁₋₆alkyl is substituted with an amino group, methyl amine, or a heterocycle.

In certain embodiments, the heterocycle is a heterocycloC₃₋₆alkyl. In certain embodiments, the heterocycle is substituted. In certain embodiments, the heterocycle contains at least one N. In certain embodiments, the heterocycle contains one or two N. In certain embodiments, the heterocycle is an azetidine, pyrrolidine, piperidine, or a piperazine. In certain embodiments, the heteroaryl is a pyridine. In certain embodiments, the heterobicyclic group contains at least one N. In certain embodiments, the heterobicyclic group is a cyclopropane fused to a piperdine.

In certain embodiments, R¹ is C═O, SO₂-Me, or sulfonyl.

In certain embodiments R² is optional, and if present, is CF₃, aryl (optionally 6 membered), substituted aryl (optionally 6 membered), benzyl, or a substituted naphthalene, wherein
the aryl group is substituted with one, two or three groups selected from: halo, —OR⁷, haloalkyl, —NH₂, C₁₋₆alkylamino (such as methylamine or dimethylamine), and C₁₋₆alkyl ester; and the naphthalene is substituted with a C₁₋₆alkylamino (optionally dimethyl amino).

In certain embodiments, R¹ is sulfonyl and R² is selected from any one of the following:

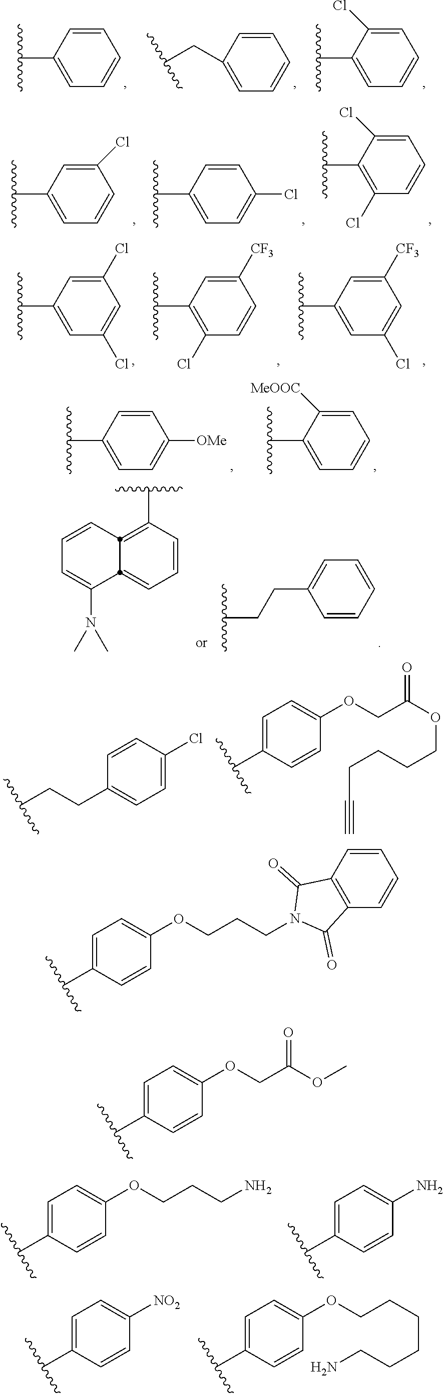

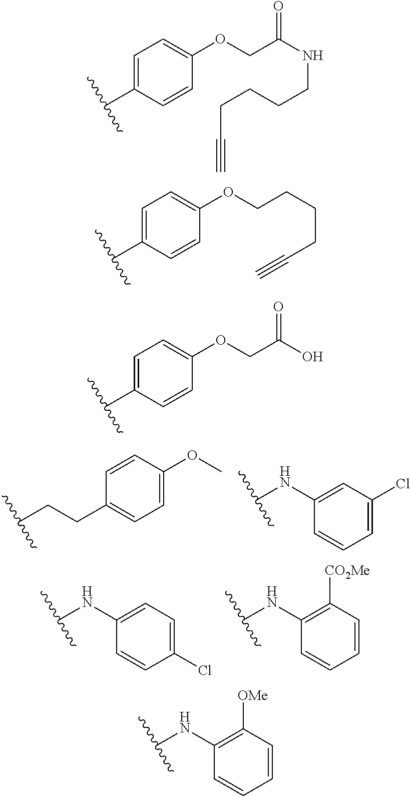

In certain embodiments, R⁷ is selected from: C₁₋₆alkynyl, —CH₂C(O)OH, —CH₂C(O)O(CH₂)ₙCH₃, —CH₂C(O)O(CH₂)ₙCCH, —CH₂C(O)NH(CH₂)ₙCH₃, —(CH₂)ₙNH₂, —(CH₂)ₙNO₂, and —(CH₂)ₙR⁹,

In certain embodiments, R⁸ is phenyl substituted with —Cl, —CO₂Me, and —OMe.

In certain embodiments, R³ is a substituted amino, wherein the amino is substituted with H or Me and a group selected from: H, C₁₋₆alkyl, —(CH₂)ₘNH₂, unsubstituted 6 membered heteroaryl, unsubstituted —(CH₂)ₘ-4 to 6-membered heterocycloalkyl or the two substituents on the amino may form a substituted or unsubstituted 5 or 6 membered ring,
wherein each group that may be substituted is substituted with one or two C₁₋₆alkyl groups.

In certain embodiments R³ is selected from:

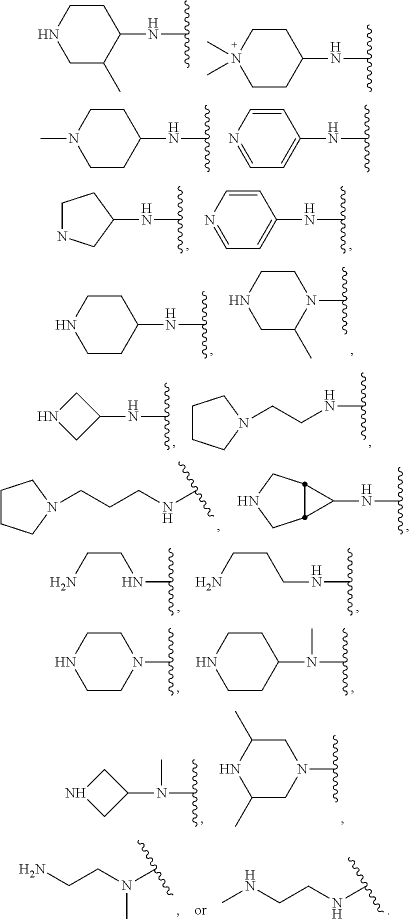

In certain embodiments R⁴ is H or a substituted amino, wherein the amino group is —C(O)aryl wherein the aryl group is substituted with R¹⁰.

In certain embodiments R¹⁰ is selected from: —OC₁₋₆alkyl, halo, —CH₂morpholinyl and diaziridine substituted with C₁₋₆haloalkyl. In certain embodiments R¹⁰ is selected from: —CF₃, Cl, NO₂, OMe, F, —CH₂morpholinyl, and diaziridine substituted with CF₃.

In certain embodiments, R⁴ is selected from:

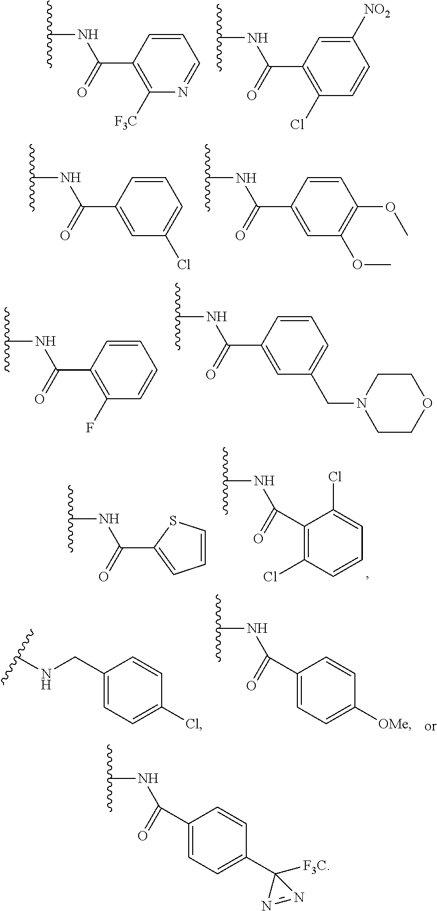

In certain embodiments, the disclosure relates to a compound of Formula II:

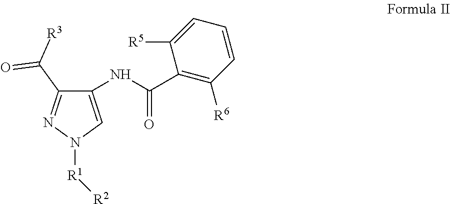

or a salt, ester, or solvate thereof, wherein R¹ is C═O, SO₂-Me, carbonyl, or a sulfonyl; wherein
R¹ is C═O, SO₂-Me, carbonyl, or a sulfonyl;
R² is optional, and if present, is CF₃, C₁₋₆alkyl, substituted C₁₋₆alkyl, amino, alkoxycarbonyl, aryl, substituted aryl, benzyl, or a substituted naphthalene, wherein
   the C₁₋₆alkyl is substituted with phenyl substituted with —OC₁₋₆alkyl,
   the aryl group is substituted with one, two or three groups selected from: halo, —OR⁷, haloalkyl, —NH₂, C₁₋₆alkylamino (such as methylamine or dimethylamine), and C₁₋₆alkyl ester;
   the naphthalene is substituted with a C₁₋₆alkylamino;
   the amino is selected from: —NHR⁸;
R³ is a substituted amino, wherein the amino is substituted with two groups (optionally where 1 group is H or Me) selected from: H, C₁₋₆alkyl, —(CH₂)ₘNH₂, substituted or unsubstituted 6 membered heteroaryl, substituted or unsubstituted —(CH₂)ₘ-4 to -6-membered heterocycloalkyl or the two substituents on the amino may form a substituted or unsubstituted 5 or 6 membered ring,
   wherein each group that may be substituted is substituted with one or two C₁₋₆alkyl groups;
R⁴ is H or a substituted amino, wherein the amino group is substituted with benzyl, —C(O)aryl or —C(O)heteroaryl wherein the benzyl group and the aryl group are unsubstituted or substituted with R¹⁰;
R⁵ and R⁶ are independently H or a halo.
R⁷ is selected from: C₁₋₆alkyl, C₁₋₆alkynyl, —CH₂C(O)OH, —CH₂C(O)O(CH₂)ₙCH₃, —CH₂C(O)O(CH₂)ₙCCH, —CH₂C(O)NH(CH₂)ₙCH₃, —(CH₂)ₙNH₂, —(CH₂)ₙNO₂, and —(CH₂)ₙR⁹,
R⁸ is phenyl substituted with halo, —CO₂H, —CO₂—C₁₋₆alkyl, and —OC₁₋₆alkyl;
R⁹ is phthalimide;
R¹⁰ is selected from: C₁₋₆alkyl, —OC₁₋₆alkyl, C₁₋₆haloalkyl, halo, —NO₂, —CH₂morpholinyl and diaziridine substituted with C₁₋₆haloalkyl; and
n is 0 to 6
m is 0 to 4.

In certain embodiments, the aryl is substituted with at least one of a halo group, OMe, CF₃, amino, and COOMe. In certain embodiments, the aryl is substituted with at least one halo group. In certain embodiments, the aryl is substituted with 1-3 halo groups. In certain embodiments, the aryl is substituted with 1 halo group, 2 halo groups, or 3 halo groups. In certain embodiments, the halo group is Cl.

In certain embodiments, the substituted naphthalene is substituted with an amino.

In certain embodiments, the amino is substituted with H, SO₂-Me, C₁₋₆alkyl, substituted C₁₋₆alkyl, carbonyl, heterocycle, heteroaryl, benzoyl, substituted benzoyl, substituted benzyl, heterobicycle, or both hydrogens together are replaced with an alkylene group (to form a ring which contains the nitrogen and optionally at least one additional nitrogen). In certain embodiments, the substituted benzoyl is substituted with at least one of a halo group, OMe, CF₃, amino, and COOMe. In certain embodiments, the substituted benzoyl is substituted with at least one halo group. In certain embodiments, the substituted benzoyl is substituted with 1-3 halo groups. In certain embodiments, the substituted benzoyl is substituted with 1 halo group, 2 halo groups, or 3 halo groups. In certain embodiments, the halo group is Cl.

In certain embodiments, the substituted C₁₋₆alkyl is substituted with an amino group, methyl amine, or a heterocycle.

In certain embodiments, the heterocycle is a heterocycloC₃₋₆alkyl. In certain embodiments, the heterocycle is substituted. In certain embodiments, the heterocycle contains at least one N. In certain embodiments, the heterocycle contains one or two N. In certain embodiments, the heterocycle is an azetidine, pyrrolidine, piperidine, or a piperazine. In certain embodiments, the heteroaryl is a pyridine. In certain embodiments, the heterobicyclic group contains at least one N. In certain embodiments, the heterobicyclic group is a cyclopropane fused to a piperdine.

In certain embodiments, R¹ is C═O, SO₂-Me, or sulfonyl.

In certain embodiments, R² is selected from any one of the following:

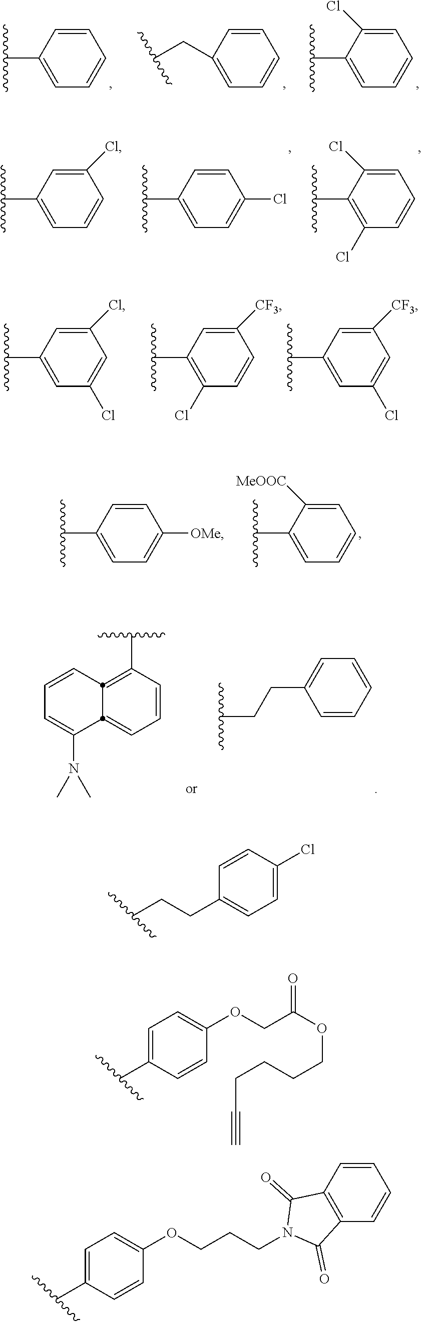

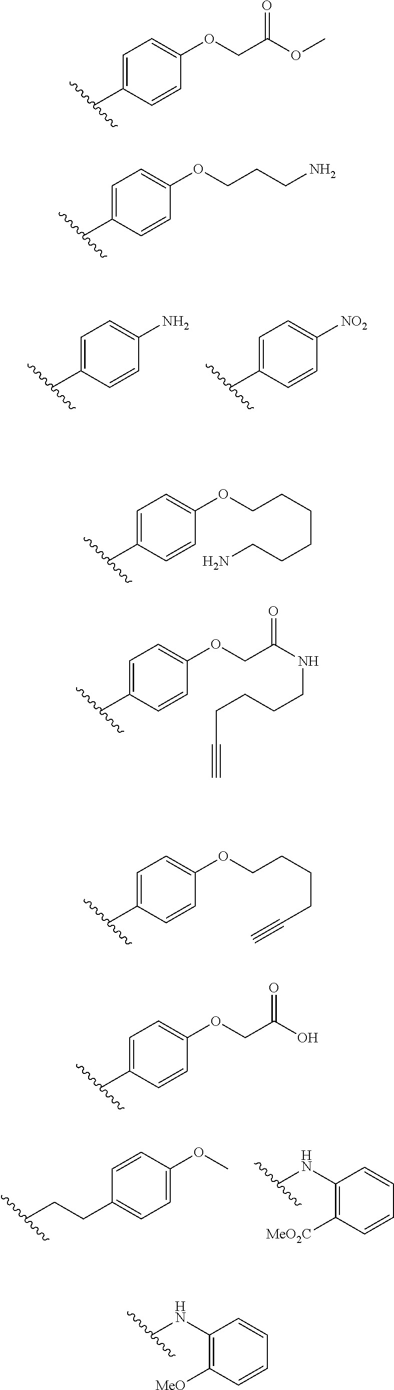

In certain embodiments R³ is

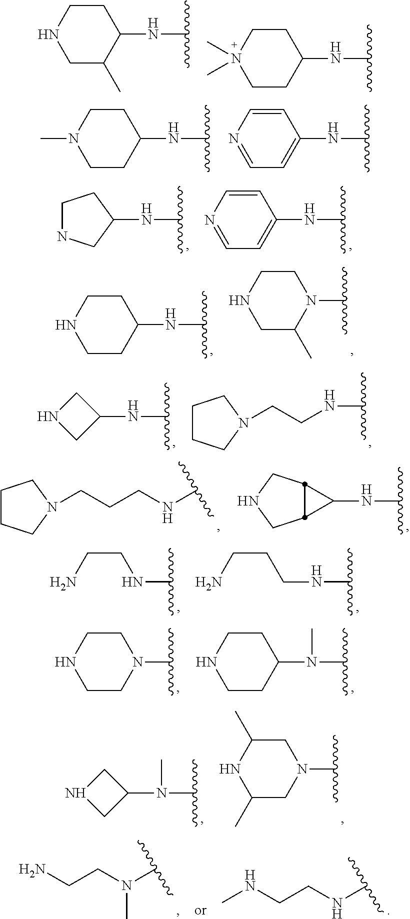

In certain embodiments, the disclosure relates to a compound of Formula III:

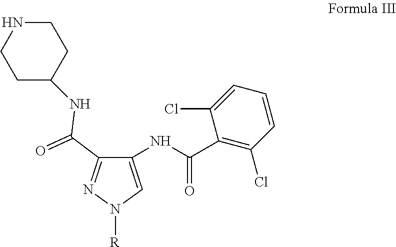

or a salt, ester, or solvate thereof, wherein R is SO₂-Me, benzoyl, substituted benzoyl, or a sulfonyl.

In certain embodiments R is a SO₂-Me or CO-2,6-dichlorobenzoyl.

In certain embodiments R is —R₁-R₂ and may take the definition of R1 and R2 set out anywhere herein.

In certain embodiments, the compound of Formulas I-III is:

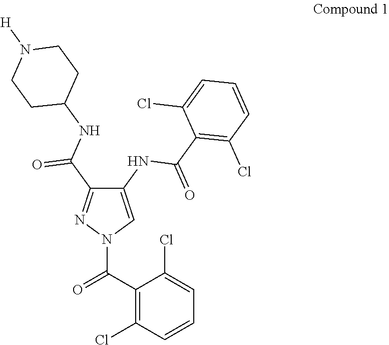 [SMILES: O=C(NC1CCNCC1)c1nn(C(=O)c2c(Cl)cccc2Cl)cc1NC(=O)c1c(Cl)cccc1Cl]

or a salt, ester, or solvate thereof.

In certain embodiments, the compound of Formulas I-III is:

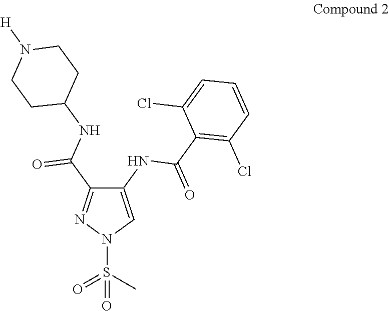 [SMILES: CS(=O)(=O)n1cc(NC(=O)c2c(Cl)cccc2Cl)c(C(=O)NC2CCNCC2)n1]

or a salt, ester, or solvate thereof.

In certain embodiments, the disclosure relates to compounds including one or more of:

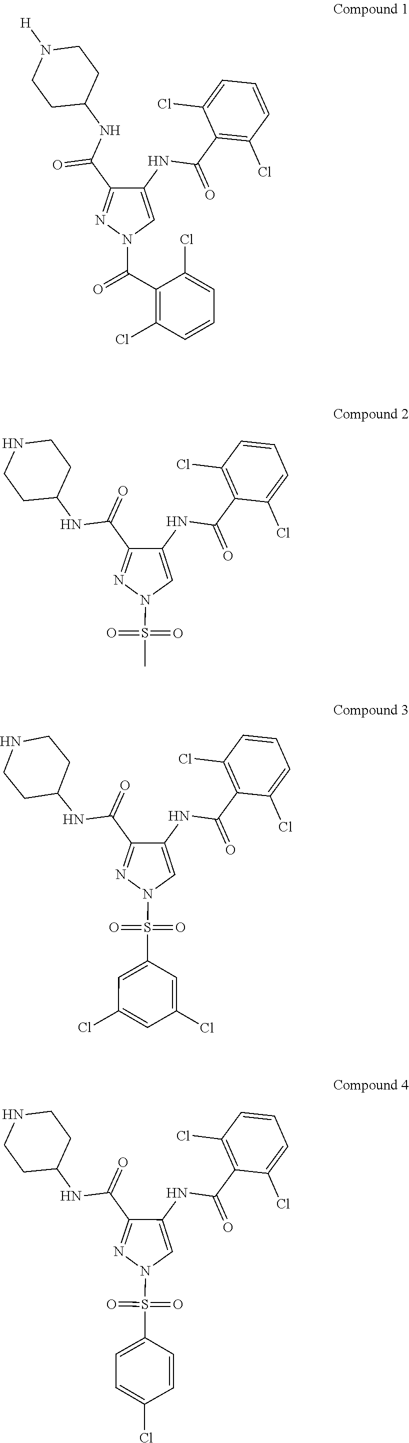

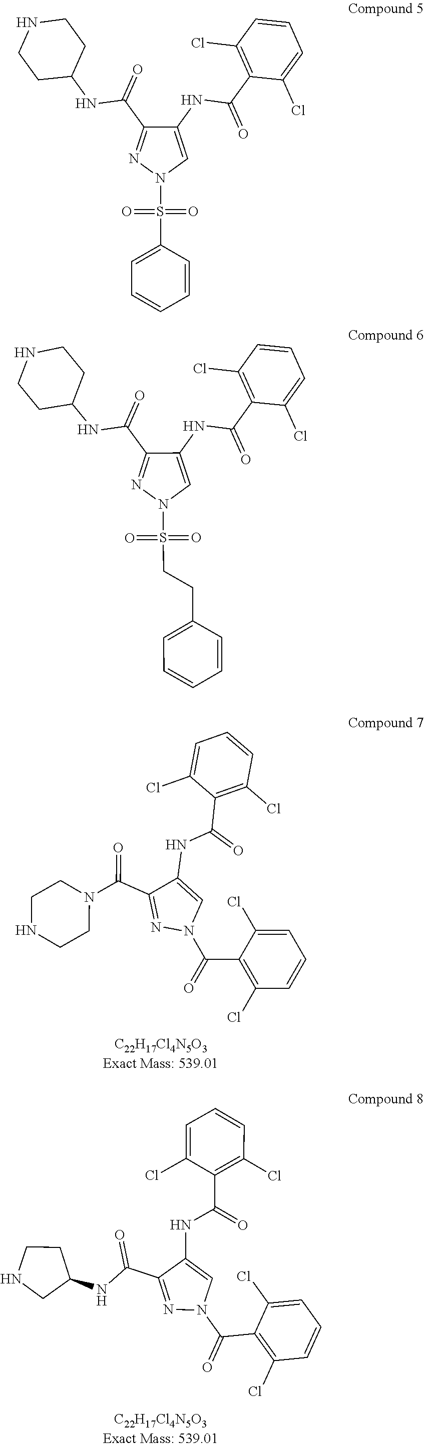 [SMILES: O=C(NC1CCNCC1)c1nn(S(=O)(=O)c2ccccc2)cc1NC(=O)c1c(Cl)cccc1Cl ; O=C(NC1CCNCC1)c1nn(S(=O)(=O)CCc2ccccc2)cc1NC(=O)c1c(Cl)cccc1Cl ; O=C(Nc1cn(C(=O)c2c(Cl)cccc2Cl)nc1C(=O)N1CCNCC1)c1c(Cl)cccc1Cl ; O=C(N[C@@H]1CCNC1)c1nn(C(=O)c2c(Cl)cccc2Cl)cc1NC(=O)c1c(Cl)cccc1Cl]

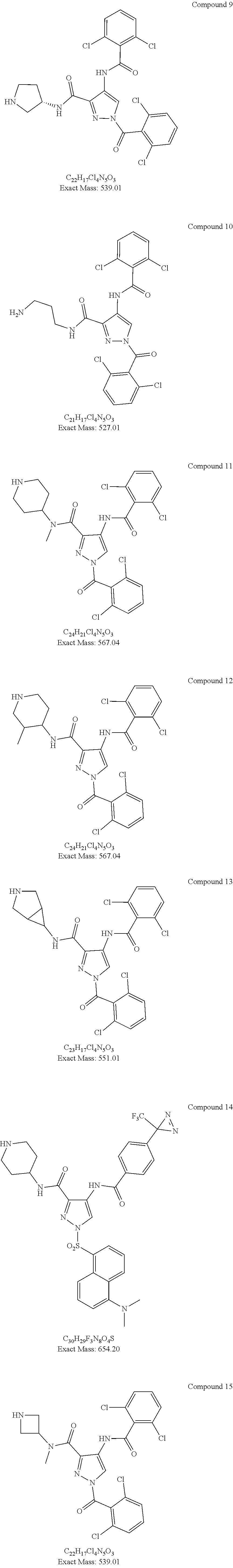

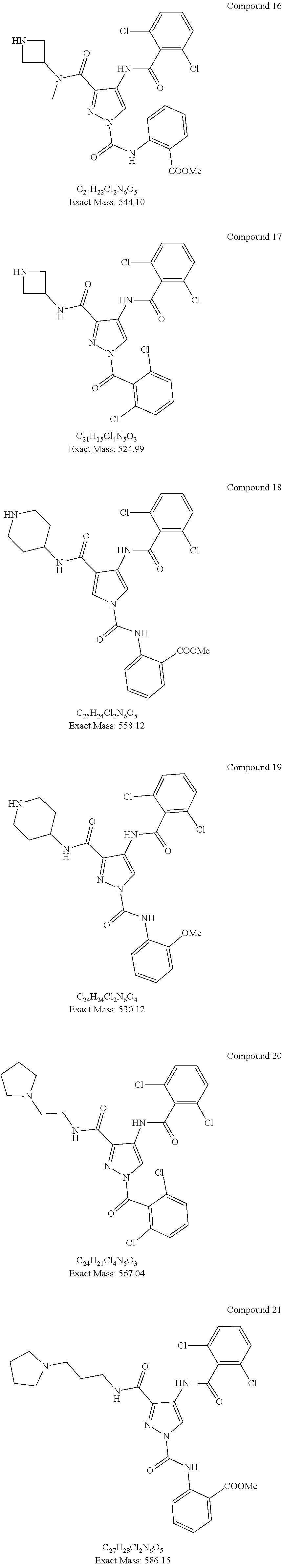 [SMILES: COC(=O)c1ccccc1NC(=O)n1cc(NC(=O)c2c(Cl)cccc2Cl)c(C(=O)N(C)C2CNC2)n1 ; O=C(NC1CNC1)c1nn(C(=O)c2c(Cl)cccc2Cl)cc1NC(=O)c1c(Cl)cccc1Cl ; COC(=O)c1ccccc1NC(=O)n1cc(NC(=O)c2c(Cl)cccc2Cl)c(C(=O)NC2CCNCC2)c1 ; COc1ccccc1NC(=O)n1cc(NC(=O)c2c(Cl)cccc2Cl)c(C(=O)NC2CCNCC2)n1 ; O=C(NCCN1CCCC1)c1nn(C(=O)c2c(Cl)cccc2Cl)cc1NC(=O)c1c(Cl)cccc1Cl ; COC(=O)c1ccccc1NC(=O)n1cc(NC(=O)c2c(Cl)cccc2Cl)c(C(=O)NCCCN2CCCC2)n1]

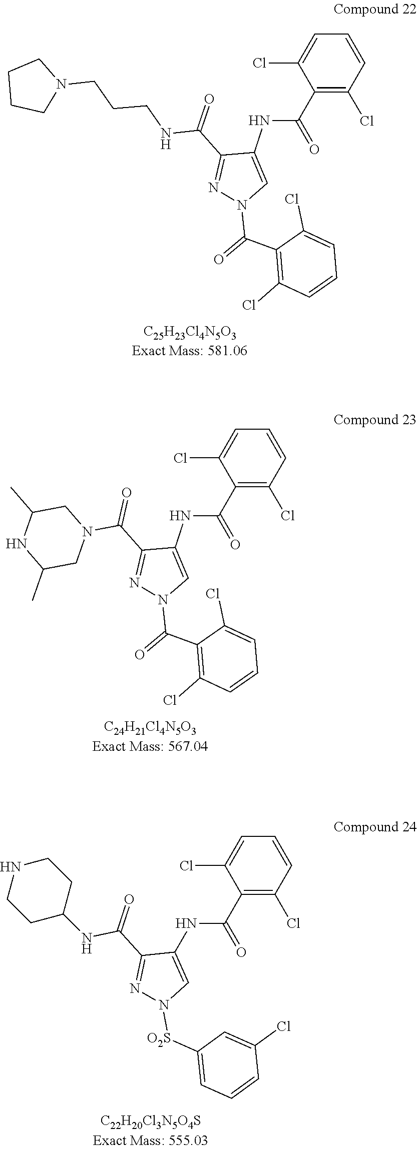 [SMILES: O=C(NCCCN1CCCC1)c1nn(C(=O)c2c(Cl)cccc2Cl)cc1NC(=O)c1c(Cl)cccc1Cl ; CC1CN(C(=O)c2nn(C(=O)c3c(Cl)cccc3Cl)cc2NC(=O)c2c(Cl)cccc2Cl)CC(C)N1 ; O=C(NC1CCNCC1)c1nn(S(=O)(=O)c2cccc(Cl)c2)cc1NC(=O)c1c(Cl)cccc1Cl]

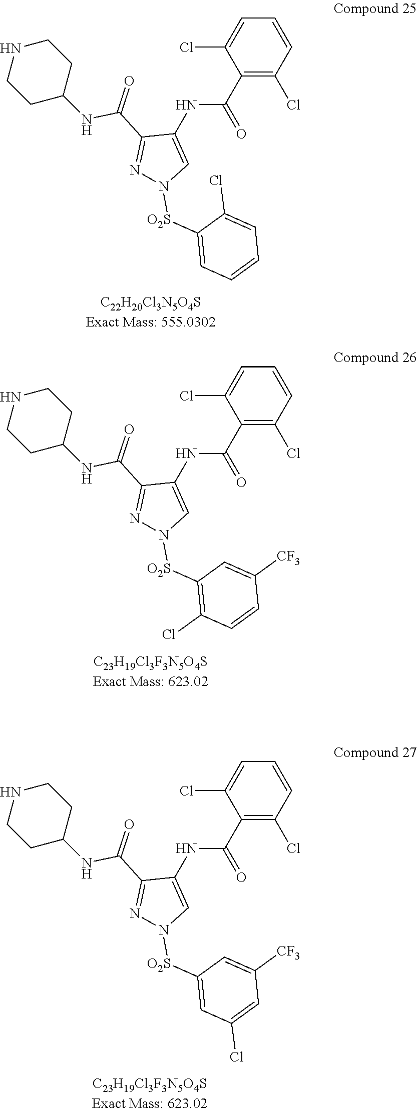 [SMILES: O=C(NC1CCNCC1)c1nn(S(=O)(=O)c2ccccc2Cl)cc1NC(=O)c1c(Cl)cccc1Cl ; O=C(NC1CCNCC1)c1nn(S(=O)(=O)c2cc(C(F)(F)F)ccc2Cl)cc1NC(=O)c1c(Cl)cccc1Cl ; O=C(NC1CCNCC1)c1nn(S(=O)(=O)c2cc(Cl)cc(C(F)(F)F)c2)cc1NC(=O)c1c(Cl)cccc1Cl]

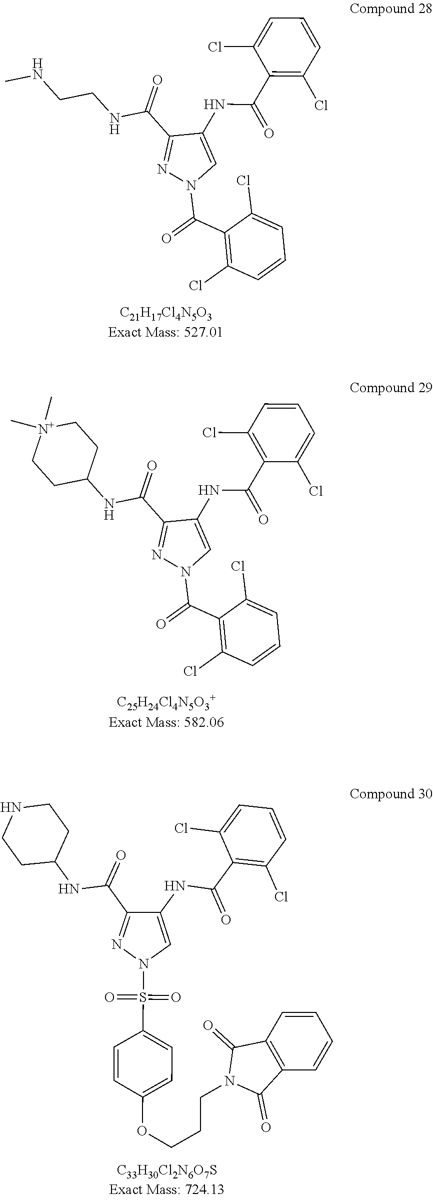 [SMILES: CNCCNC(=O)c1nn(C(=O)c2c(Cl)cccc2Cl)cc1NC(=O)c1c(Cl)cccc1Cl ; C[N+]1(C)CCC(NC(=O)c2nn(C(=O)c3c(Cl)cccc3Cl)cc2NC(=O)c2c(Cl)cccc2Cl)CC1 ; O=C(NC1CCNCC1)c1nn(S(=O)(=O)c2ccc(OCCCN3C(=O)c4ccccc4C3=O)cc2)cc1NC(=O)c1c(Cl)cccc1Cl]

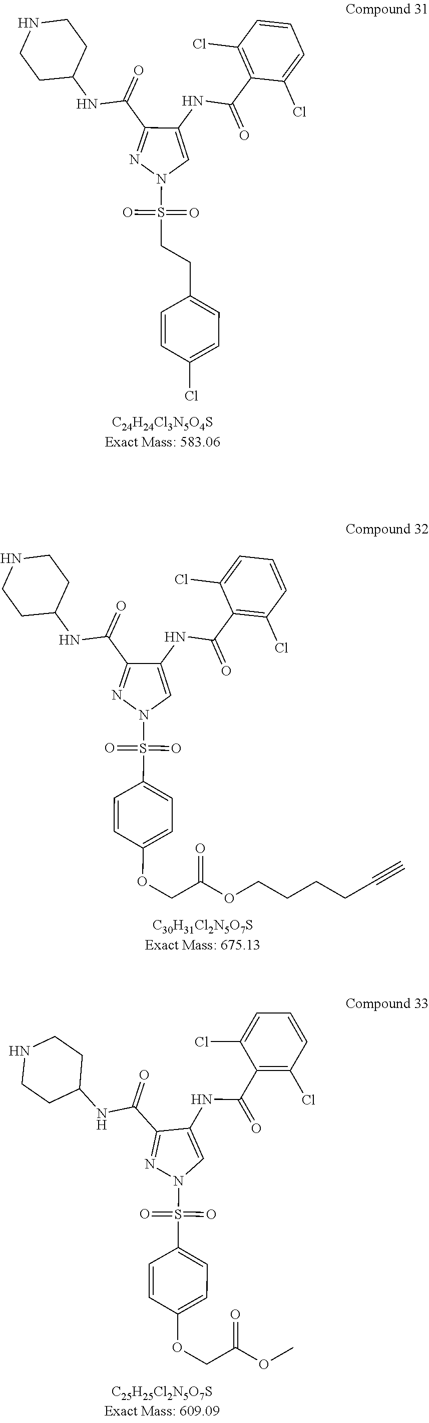 [SMILES: O=C(NC1CCNCC1)c1nn(S(=O)(=O)CCc2ccc(Cl)cc2)cc1NC(=O)c1c(Cl)cccc1Cl ; C#CCCCCOC(=O)COc1ccc(S(=O)(=O)n2cc(NC(=O)c3c(Cl)cccc3Cl)c(C(=O)NC3CCNCC3)n2)cc1 ; COC(=O)COc1ccc(S(=O)(=O)n2cc(NC(=O)c3c(Cl)cccc3Cl)c(C(=O)NC3CCNCC3)n2)cc1]

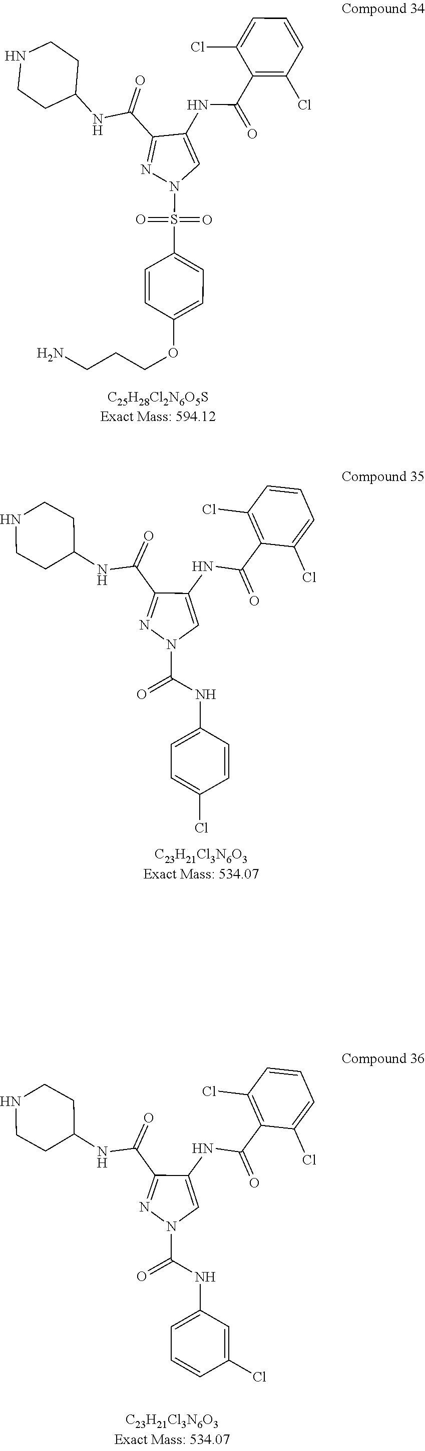 [SMILES: NCCCOc1ccc(S(=O)(=O)n2cc(NC(=O)c3c(Cl)cccc3Cl)c(C(=O)NC3CCNCC3)n2)cc1 ; O=C(NC1CCNCC1)c1nn(C(=O)Nc2ccc(Cl)cc2)cc1NC(=O)c1c(Cl)cccc1Cl ; O=C(NC1CCNCC1)c1nn(C(=O)Nc2cccc(Cl)c2)cc1NC(=O)c1c(Cl)cccc1Cl]

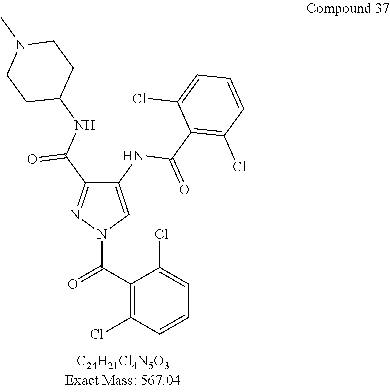 [SMILES: CN1CCC(NC(=O)c2nn(C(=O)c3c(Cl)cccc3Cl)cc2NC(=O)c2c(Cl)cccc2Cl)CC1]

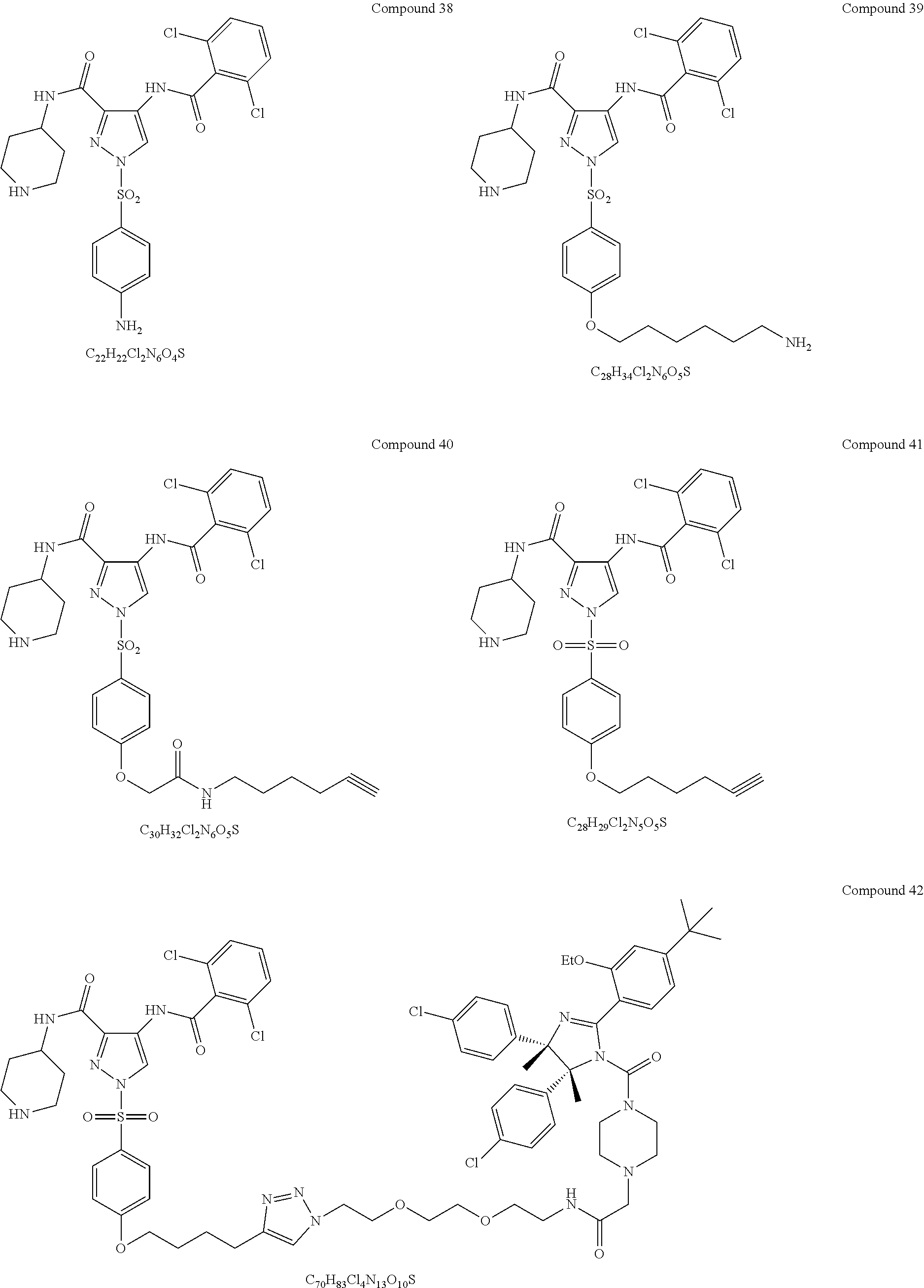

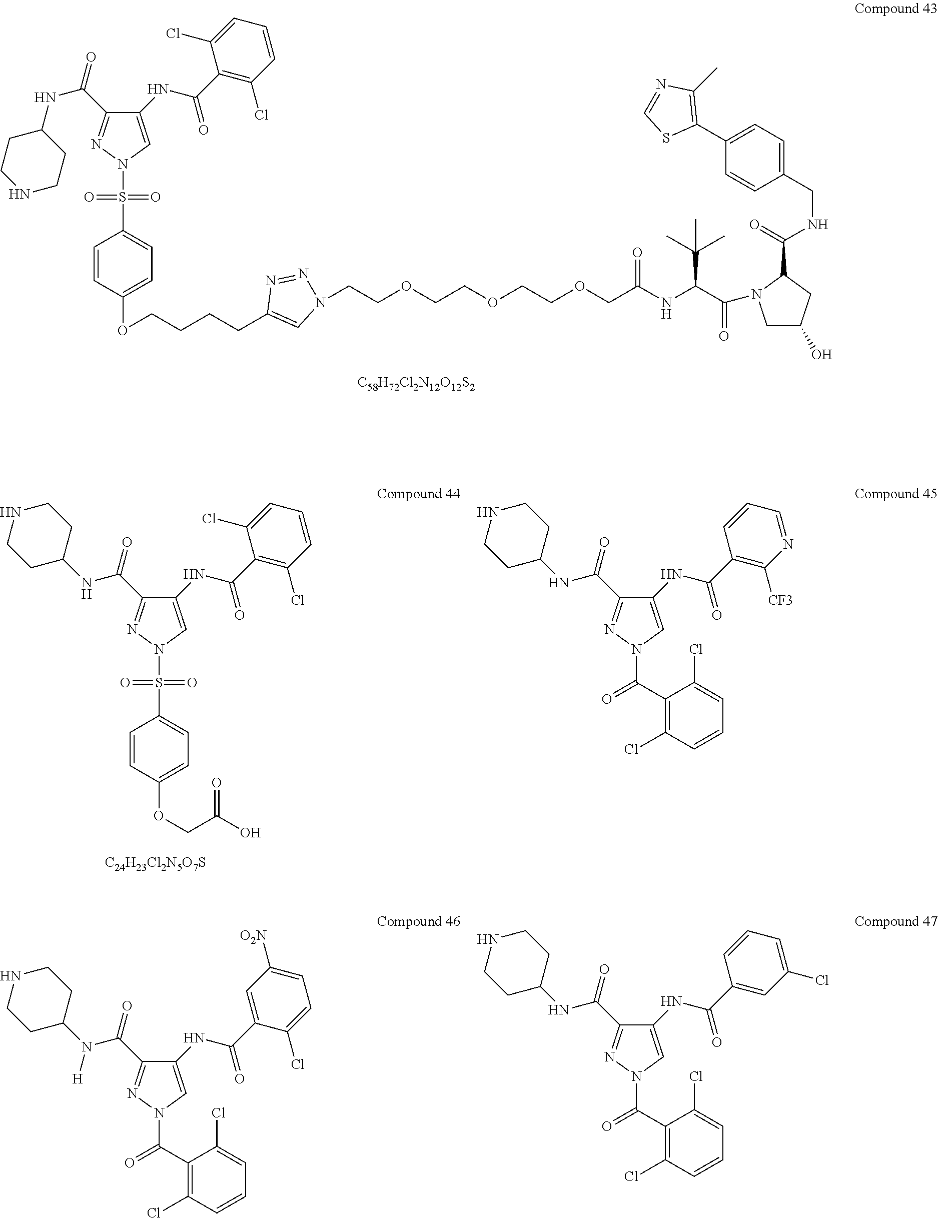

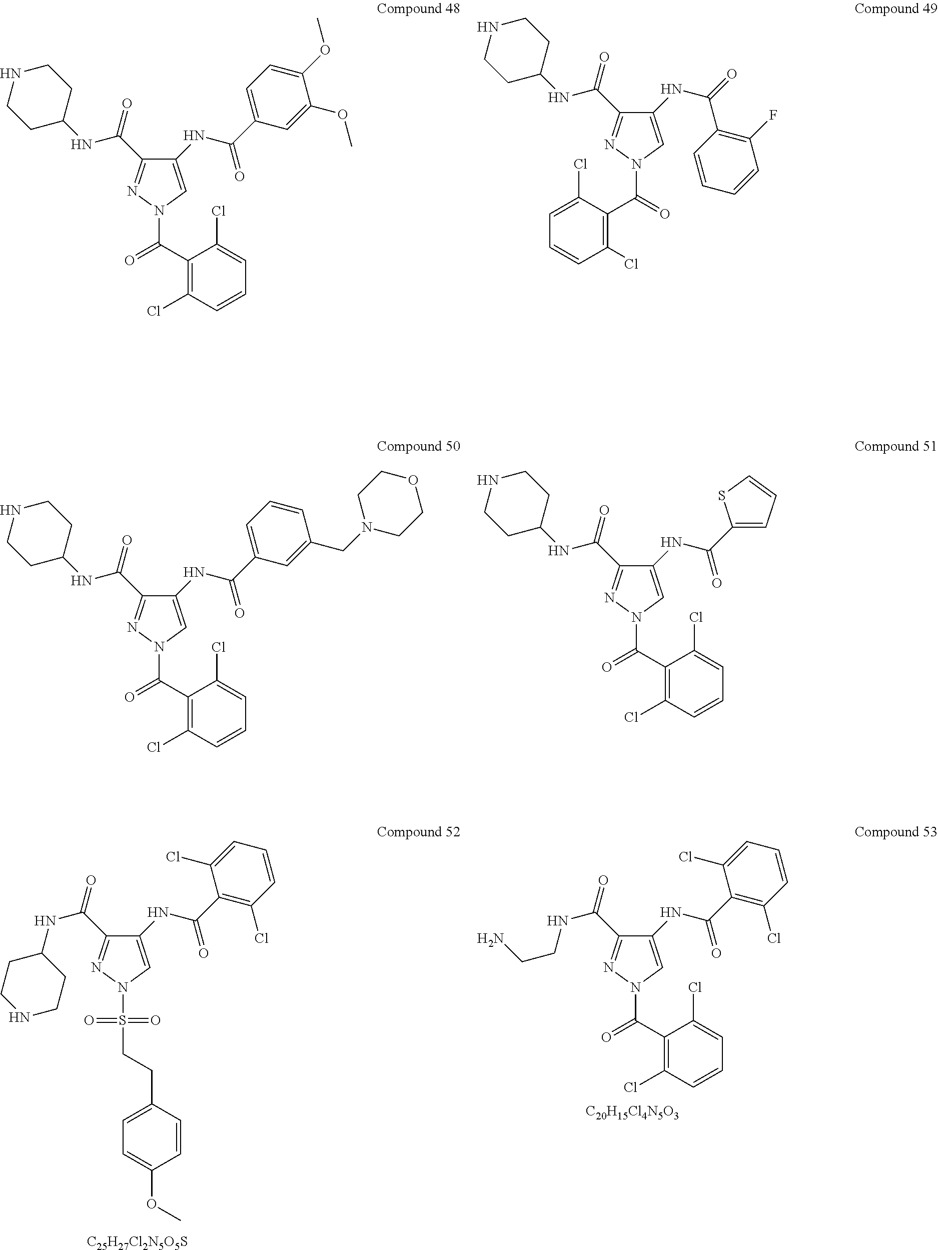

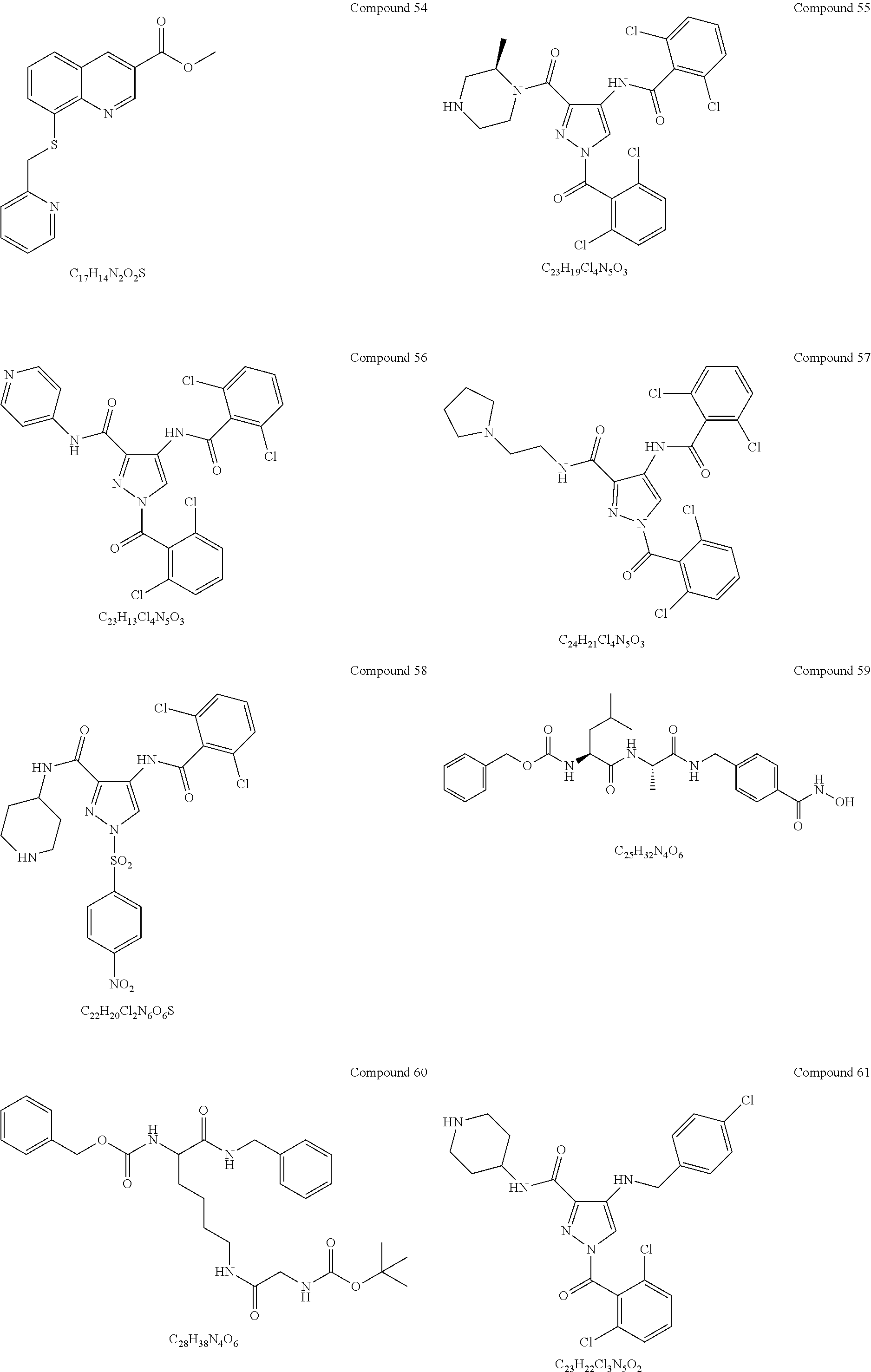

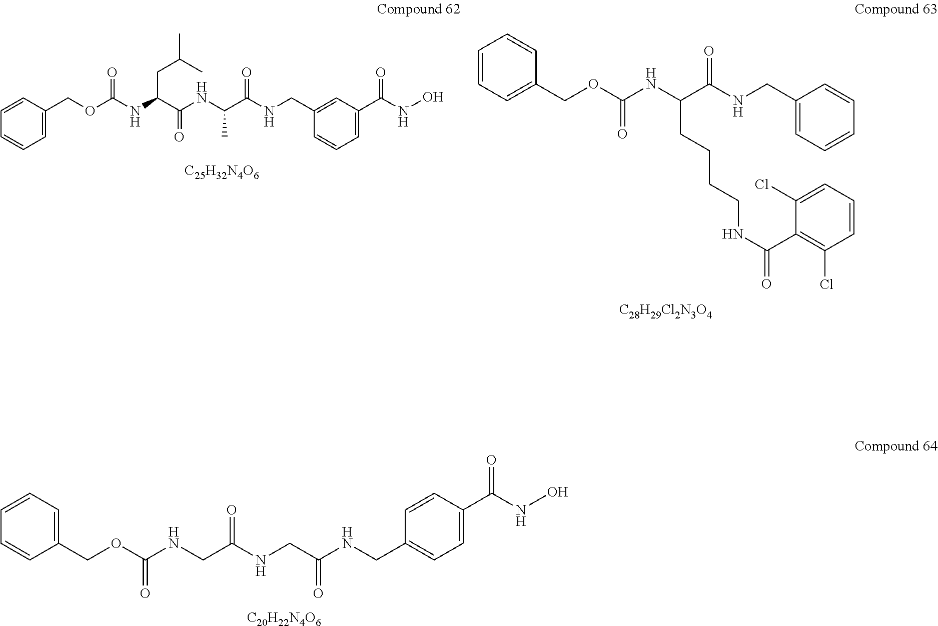

or a salt, ester, or solvate thereof.

Acceptable salts include, but are not limited to, those prepared from the following acids: hydrochloric, hydrobromic, sulphuric, nitric, phosphoric, maleic, salicylic, p-toluenesulfonic, tartaric, citric, methanesulphonic, formic, malonic, succinic, naphthalene-2-sulphonic, and benzenesulphonic. Also, acceptable salts can be prepared as alkaline metal or alkaline earth salts, such as sodium, potassium or calcium salts of the carboxylic acid group.

### 2. Exemplary Methods

In certain embodiments, methods of inhibiting BRISC enzymes are provided and comprise contacting BRISC enzymes with an effective amount of at least one compound according to Formulas I-III. In certain embodiments, methods of inhibiting type I interferon receptor (IFNAR1) signaling are provided and comprise contacting BRISC enzymes with an effective amount of at least one compound according to Formula I-III. In certain embodiments, the compound is Compound 1 or Compound 2. These methods can involve either an assay or a method of treatment of a subject, e.g., a human subject, including administering to the subject a therapeutically effective amount of a compound of Formulas I-III.

In certain embodiments, methods of treating or preventing an autoimmune disorder are provided and comprise administering an effective amount of at least one compound according to Formula I-III. In certain embodiments, the compound is Compound 1 or Compound 2.

Examples of autoimmune disorders include, but are not limited to, Hashimoto's thyroiditis, pernicious anemia, Addison's disease, type 1 diabetes, rheumatoid arthritis, systemic lupus erythematosus, scleroderma, Sjogren's syndrome, dermatomyositis, lupus erythematosus, multiple sclerosis, autoimmune inner ear disease myasthenia gravis, Reiter's syndrome, Graves disease, autoimmune hepatitis, familial adenomatous polyposis, and ulcerative colitis

In certain embodiments, methods of treating or preventing scleroderma are provided and comprise administering an effective amount of at least one compound according to Formula I-III. In certain embodiments, the compound is Compound 1 or Compound 2.

In certain embodiments, methods of treating or preventing SLE are provided and comprise administering an effective amount of at least one compound according to Formula I-III. In certain embodiments, the compound is Compound 1 or Compound 2.

In certain embodiments, methods of treating or preventing an inflammatory disease are provided and comprise administering an effective amount of at least one compound according to Formula I-III. In certain embodiments, the compound is Compound 1 or Compound 2.

Examples of the inflammatory disease include, but are not limited to, metabolic syndrome, allergy, renal disease, infection, autoimmune disease, rheumatoid arthritis, multiple sclerosis, organ transplant rejection, and cancer metastasis. Examples of the metabolic syndrome include insulin resistance, hyperinsulinemia, type 2 diabetes mellitus, hyperlipidemia, arteriosclerosis, hypertension, obesity, and visceral fat accumulation.

As used herein, a “therapeutically effective amount” refers to an amount of the compound sufficient to treat, prevent, or manage the disease (e.g., SLE). A therapeutically effective amount can refer to the amount of a compound that provides a therapeutic benefit in the treatment or management of the disease (e.g., SLE). In certain embodiments, a therapeutically effective amount can be an amount to treat, for example, an SLE flare up. In certain embodiments, a therapeutically effective amount can be an amount to treat the disease (e.g., SLE) chronically. Further, a therapeutically effective amount with respect to a BRISC inhibitor of the disclosure can mean the amount of therapeutic alone, or in combination with other therapies, that provides a therapeutic benefit in the treatment or management of the disease (e.g., SLE), which can include a decrease in severity of disease symptoms (e.g., in the case of SLE: fatigue, fever, joint pain, joins stiffness, joint swelling, skin rashes, skin lesions, Raynaud's phenomenon, shortness of breath, chest pain, dry eyes, headaches, confusion, memory loss, and difficulty urinating), an increase in frequency and duration of disease symptom-free periods, or a prevention of impairment or disability due to the disease affliction. The term can encompass an amount that improves overall therapy, reduces or avoids unwanted effects, or enhances the therapeutic efficacy of or synergies with another therapeutic agent.

Animal models accepted in the art as models of disease (e.g., SLE) can be used to test particular peptide compounds, routes of administration etc., to determine appropriate amounts of therapeutic treatments of the disclosure.

The ability of a therapeutic to inhibit disease (e.g., SLE) can be evaluated in an animal model system predictive of efficacy in a human. Alternatively, this property of a therapeutic can be evaluated by examining the ability of the therapeutic to decrease of type I interferon receptor (IFNAR1) signaling, in addition to TLR4 and TLR7 signaling.

In certain embodiments, a composition of the present disclosure can be administered via one or more routes of administration using one or more of a variety of methods known in the art. As will be appreciated by the skilled artisan, the route and/or mode of administration will vary depending upon the desired results. Routes of administration for the BRISC inhibitor of this disclosure include, but are not limited to, intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

In certain embodiments, a BRISC inhibitor of this disclosure can be administered via a non-parenteral route, such as a topical, epidermal or mucosal route of administration, for example, intranasally, orally, vaginally, rectally, sublingually or topically.

In certain embodiments, therapeutic compositions can be administered with medical devices known in the art. For example, in certain embodiments, a therapeutic composition of this disclosure can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

### 3. Pharmaceutical Compositions

The BRISC inhibitor of the disclosure can be formulated into compositions suitable for pharmaceutical administration. Pharmaceutical compositions of this disclosure also can be administered in combination therapy, i.e., combined with other agents. In certain embodiments, the combination therapy can include a BRISC inhibitor of the present disclosure combined with at least one additional agent.

In certain embodiments, the additional agent can be a corticosteroid. “Corticosteroid” is meant to include any naturally occurring or synthetic steroid hormone which can be derived from cholesterol and is characterized by a hydrogenated cyclopentanoperhydrophenanthrene ring system. Corticosteroids for use in the method of the disclosure include, but are not limited to: dexamethasone and its derivatives (e.g., dipropionate and valerate), triamcinolone and its derivatives (e.g., diacetate, hexacetonide, and acetonide), betamethasone and its derivatives (e.g., the dipropionate, benzoate, sodium phosphate, acetate, and valerate), flunisolide, prednisone and its derivatives (e.g., acetate), prednisolone and its derivatives (e.g., acetate, sodium phosphate and tebutate), methylprednisolone and its derivatives (e.g., acetate and sodium succinate), fluocinolone and its derivatives (e.g., acetonide), diflorasone and its derivatives (e.g., diacetate), halcinonide, desoximetasone (e.g., desoxymethasone), diflucortolone and its derivatives (e.g., valerate), flucloronide (e.g., fluclorolone acetonide), fluocinonide, fluocortolone, fluprednidene and its derivatives (e.g., acetate), flurandrenolide (e.g., flurandrenolone), clobetasol and its derivatives (e.g., propionate), clobetasone and its derivatives (e.g., butyrate), alclometasone, flumethasone and its derivatives (e.g., pivalate), fluocortolone and its derivatives (e.g., hexanoate), amcinonide, beclometasone and its derivatives (e.g., dipropionate), fluticasone and its derivatives (e.g., propionate), difluprednate, prednicarbate, flurandrenolide, mometasone, and desonide.

In certain embodiments, the additional agent can be a NSAID. Suitable NSAIDs useful in this disclosure include piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, and salicylates such as aspirin. The COX-2 inhibitors (such as meloxicam, celecoxib, rofecoxib, valdecoxib and etoricoxib) and the cylco-oxygenase inhibiting nitric oxide donors (CLNOD's).

Additional non-steroidal anti-inflammatory compounds useful in this disclosure include methotrexate; sulphasalazine; cyclosporine A; lefunomide; ciclesonide; hydroxychloroquine; d-penicillamine; auranofin; parenteral or oral gold; leukotriene biosynthesis inhibitors; 5-lipoxygenase (5-LO) inhibitors; 5-lipoxygenase activating protein (FLAP) antagonists selected from the group consisting of zileuton, ABT-761, fenleuton, tepoxalin, Abbott-79175, Abbott-85761, N-(5 substituted)-thiophene-2-alkylsulfonamides, 2,6-di-tert-butylphenol hydrazones, methoxytetrahydropyrans such as Zeneca ZD-2138, the compound SB-210661, pyridinyl-substituted 2n cyanonaphthalene compounds such as L-739,010, 2-cyanoquinoline compounds such as L-746,530, and indole and quinoline compounds such as MK-591, MK-886, and BAY×1005.

In certain embodiments, the additional agent can be one or more of methotrextate, Benlysta (belimumab), or other immune modulatory biologics.

As used herein the term “pharmaceutically acceptable carrier” is a pharmaceutically acceptable solvent, suspending agent or vehicle for delivering the BRISC inhibitor to the animal or human. The carrier can be liquid or solid and is selected with the planned manner of administration being used. For example, a pharmaceutically acceptable carrier is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. In certain embodiments, supplementary active compounds can also be incorporated into the compositions.

A pharmaceutical composition of the disclosure is formulated to be compatible with its intended route of administration. For example, solutions or suspensions used for parenteral, intradermal, or subcutaneous application can include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (such as water) or dispersions and sterile powders for the extemporaneous preparation of sterile injectable solutions or dispersion. For intravenous administration, suitable carriers can include physiological saline, bacteriostatic water, Cremophor EL. (BASF, Parsippany, N.J.) or phosphate buffered saline (PBS). In certain embodiments, the composition must be sterile and can be fluid to the extent that easy syringability exists. In certain embodiments, the composition must be stable under the conditions of manufacture and storage and must be preserved against the contaminating action of microorganisms such as bacteria and fungi. In certain embodiments, the carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In certain embodiments, isotonic agents, for example, sugars, polyalcohols such as manitol, sorbitol, sodium chloride can be included in the composition. Prolonged absorption of the injectable compositions can be brought about by including in the composition an agent which delays absorption, for example, aluminum monostearate and gelatin.

In certain embodiments, sterile injectable solutions can be prepared by incorporating the active compound (i.e., the BRISC inhibitor) in the required amount in an appropriate solvent with one or a combination of ingredients enumerated above, as required, followed by filtered sterilization. Dispersions can be prepared by incorporating the active compound into a sterile vehicle which contains a basic dispersion medium and the required other ingredients from those enumerated above. In the case of sterile powders for the preparation of sterile injectable solutions, methods of preparation can include vacuum drying and freeze-drying which yields a powder of the active ingredient plus any additional desired ingredient from a previously sterile-filtered solution thereof.

In certain embodiments, oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc.

In certain embodiments, liposomal suspensions (including liposomes targeted to tumor cells with monoclonal antibodies to tumor antigens) can also be used as pharmaceutically acceptable carriers. These can be prepared according to methods known to those skilled in the art, for example, as described in U.S. Pat. No. 4,522,811, and International Patent Application Serial No. PCT/US94/07327. For example, liposome formulations can be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of invariant chain protein or peptide is then introduced into the container. The container is then swirled by hand to free lipid material from the sides of the container and to disperse lipid aggregates, thereby forming the liposomal suspension.

Pharmaceutical compositions, including, but not limited to, such liposomal suspensions and other microencapsulated compositions, can be combined with targeting agents to allow for tissue specific delivery of the BRISC inhibitor of the disclosure. In certain embodiments, such targeting can be achieved, without limitation, through the use of tissue specific antibodies and antibody mimetics. Non-limiting examples of antibody mimetics include, but are not limited to, molecules such as Affibodies, DARPins, Anticalins, Avimers, and Versabodies, all of which employ binding structures that, while they mimic traditional antibody binding and therefore can be used to target peptides to tissues specifically expressing the antigen recognized by the mimetic, are generated from and function via distinct mechanisms.

Pharmaceutical compositions can also be prepared wherein the BRISC inhibitor of the disclosure is covalently or non-covalently attached to a nanoparticle. By way of example, but not limitation, a nanoparticle can be a dendrimer, such as the polyamidoamine employed in Kukowska-Latallo et al., (2005) Cancer Res., vol. 65, pp. 5317-24, which is incorporated herein by reference in its entirety. Other dendrimers that can be used in conjunction with the BRISC inhibitor of the instant disclosure include, but are not limited to, polypropylenimine dendrimers as described in U.S. Pat. No. 7,078,461, which is hereby incorporated by reference in its entirety.

In certain embodiments, the oral or parenteral compositions can be formulated in dosage unit form for ease of administration and uniformity of dosage. “Dosage unit form” as used herein refers to physically discrete units suited as unitary dosages for the subject to be treated, each unit containing a predetermined quantity of active compound calculated to produce the desired therapeutic effect in association with the required pharmaceutical carrier. The specification for the dosage unit forms of the present disclosure are dictated by and directly dependent on (a) the unique characteristics of the active compound and the particular therapeutic effect to be achieved, and (b) the limitations inherent in the art of compounding such an active compound for the treatment of individuals.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being toxic to the subject. The selected dosage level will depend upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health, and prior medical history of the patient being treated, and like factors well known in the medical arts.

### 4. Kits

Kits are also contemplated for the disclosed subject matter. In certain embodiments, a kit can include a container, such as a vial, for the BRISC inhibitor compound and/or composition and instructions, such as a product insert or label, directing the user to utilize the compound and/or composition for treating or preventing a disease in a subject.

In certain embodiments, the disease is an autoimmune disease. In certain embodiments, the disease is an inflammatory disease. In certain embodiments, the disease is SLE. In certain embodiments, the disease is scleroderma.

The present disclosure is further illustrated by the following Examples which should not be construed as further limiting.

### EXAMPLES

### Example 1: Mouse Models of Systemic Lupus Erythematosus

Lupus patients and mouse models display elevated interferon responsive gene expression, and as reported Ifnar1−/− mice are resistant to a lupus like syndrome after pristane (2,6,10,14-tetramethylpentadecane, or TMPD) administration, and several different modalities that inhibit type I interferon signals have entered clinical trials with evidence of efficacy. Accordingly, this Example investigated whether BRISC deficiency could mitigate SLE pathogenesis. Intraperitoneal (i.p.) injection of pristane in mice produces a Lupus like syndrome that exhibits characteristic anti-DNA antibodies and glomerulonephritis that results in renal dysfunction in afflicted mice.

### Materials and Methods

Antibodies. The following antibodies and other reagents were purchased from the following commercial sources: Sigma-Aldrich (M2 FLAG, anti-SHMT2), Cell Signaling (anti-pSTAT1, STAT1, tubulin, ISG15), Santa Cruz (PKR, STAT-1), Millipore (K63-Ub), and LS Biosciences (anti-KIAA0157). In-house-produced purified antibodies were also used for immunoprecipitating KIAA0157, and SHMT2 proteins. Antibodies against phosphoSer535-IFNAR1 were previously described (Kumar et al., 2004).

Mice. The KIAA0157 floxed transgenic mice, Kiaa0157−/− mice have been previously described (Zheng et al., Cell Reports 5:180-193). The Kiaa0157^{-/-}, Ifnar1^{-/-} double knockout mice were generated by breeding Kiaa0157^{-/-} and Ifnar1^{-/-} mice. The University of Pennsylvania Institutional Animal Care and Use Committee approved all animal protocols, and all procedures were performed in accordance with these protocols.

Induction of disease model. For Kiaa0157^{-/-} mouse model of bacterial septic shock, the mice were treated with LPS (10.5 mg/kg, intraperitoneal injection) and sacrificed when they became moribund and displayed the loss of righting reflex, loss of >20% of body weight, and nonresponsiveness to footpad compression as previously described (Karaghiosoff et al., 2003). Lung tissues from LPS treated wild-type and Kiaa0157 null mice were harvested at 4-5 days of injection. Lungs were inflated and fixed with 4% paraformaldehyde, dehydrated, and embedded in paraffin. Tissue were further sectioned and stained with hematoxylin and eosin. Levels of IL1b protein in blood plasma were analyzed using ELISA kit (eBioscience).

Intraperitoneal injection of tetramethylpentadecane (pristane) was performed in 6-8 week old mice. Mice were examined at 2 weeks and 6 months after injection for markers of acute and chronic lupus, respectively. Kiaa0157 null mice showed reduced markers of peritoneal cell invasion at 2 weeks after injection and reduced autoantibodies and renal pathology at 6 months.

### Results and Discussion

BRISC deubiquitinates actively engaged Type 1 interferon receptor (IFNAR1) in both human and mouse cells to promote signaling downstream of interferon-α and -β (FIGS. 2A, B). Mouse embryonic fibroblasts were derived from embryonic day 13.5 embryos. IFNAR1 can become hyper K63-ubiquitinated in cells that have a targeted deletion of the BRISC specific subunit, Kiaa0157, which is required for BRISC stability and DUB activity. Kiaa0157^{-/-} cells can show more rapid IFNAR1 internalization and degradation in the lysosome following interferon treatment. As a consequence, BRISC deficiency can mitigate interferon signaling, tissue damage, and mortality in mice exposed to lipopolysaccharide (LPS) (FIG. 2), suggesting that BRISC DUB inhibition could be a novel target for diseases arising from elevated interferon signaling.

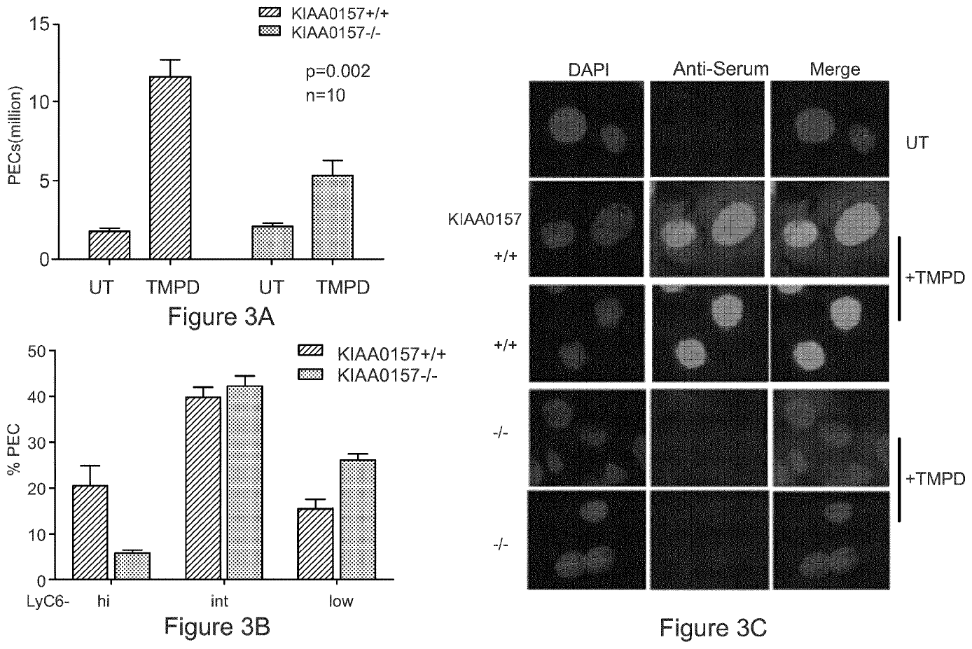

Kiaa0157^{-/-} and Kiaa0157 mice were injected with pristane, and monitored at 2 week and 6 month intervals. BRISC null mice showed a significant reduction in peritoneal inflammatory cell invasion at 2 weeks after pristane (FIG. 3A) and reduced lung hemorrhage compared to wild type controls (7 of 12 wild type mice and 1 of 12 Kiaa0157^{-/-}). Moreover, they demonstrated minimal autoantibodies to chromatin in comparison to wild type counterparts (FIGS. 3B,C). Sera from 17 of 32 wild type mice displayed immunoblot positivity against chromatin fractions from human cells and anti-nuclear antibodies by immunofluorescence compared to 5 of 33 Kiaa0157−/− mice at 6 months after pristane injection (p<0.002). Importantly, BRISC null mice exhibited reduced complement and IgG deposition in glomeruli, as well as less glomerular cellularity, architectural changes and proteinuria in comparison to wild type mice (p<0.001).

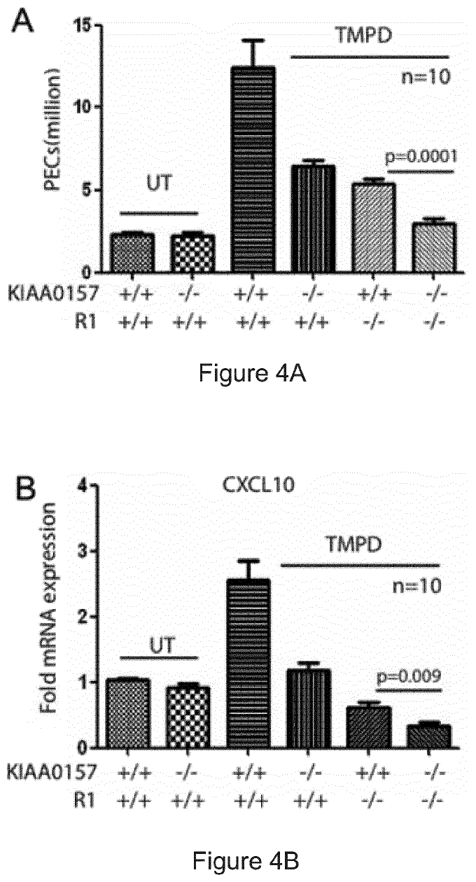

Given the central importance of K63-Ub to cytokine signaling, it was determined whether BRISC inhibition would affect multiple aspects of inflammatory signaling (FIG. 1). Consequently, small molecule BRISC inhibition can offer advantages over specifically targeting a single cytokine receptor, as the etiology of SLE is likely to be multifactorial. In agreement, Kiaa0157^{-/-} Ifnar1^{-/-} double knockout mice showed greater reductions in inflammatory cytokine responsive gene expression than either knockout alone (FIG. 4). This suggests the existence of BRISC substrates beyond Ifnar1.

### Example 2: Development of BRISC Inhibitors

Potent and specific BRISC DUB inhibitors were derived from high throughput screening and structure-based rational drug design. This is the first high throughput screen for the identification of BRISC inhibitors.

### Materials and Methods

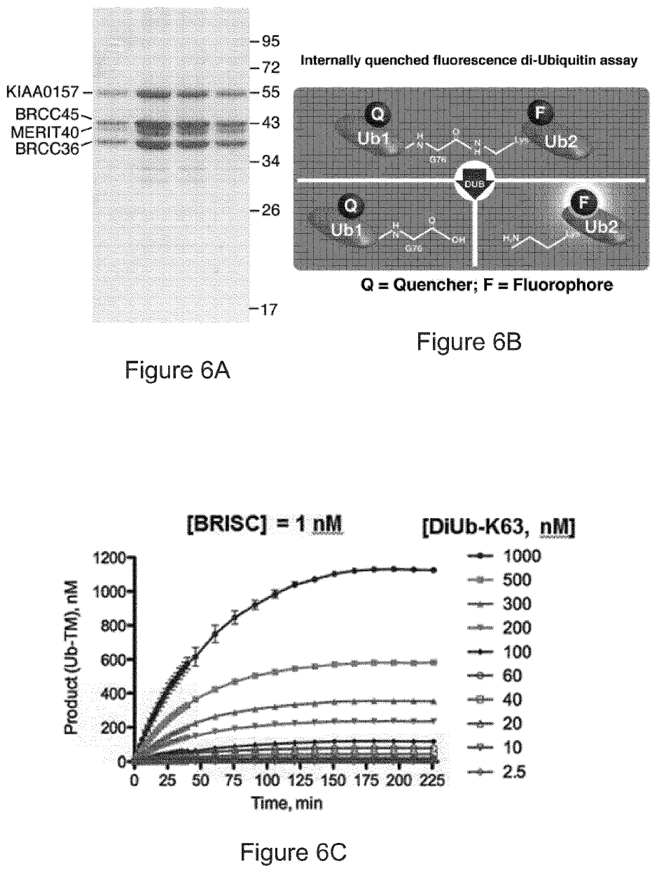

High throughput screening. A baculoviral expression system was established to purify milligram quantities of the entire BRISC complex from insect cells. The MultiBac system was used to clone all 4 components of the BRISC complex into one vector which was then packaged into the baculovirus genome using the methods described in Zeqiraj et al., 2015 (Mol. Cell). This complex exhibited robust activity against a commercially available quenched K63-linked di-ubiquitin substrate, forming the basis for high throughput screens (FIGS. 6A,B,E).

The assay buffer included: 50 mM HEPES-NaOH, pH 7.0; 100 mM NaCl; 1 mM DTT; 1 mg/ml BSA; and 0.03% Brij-35. BRISC was assayed at 1 nM (final concentration) with the substrate at 500 nM (final concentration). The final reaction volume was 20 μl in each 384 plate well (Corning, #3573; black, low flange, flat bottom) with a final concentration of compounds of 10 μM.

A 5 ml of enzyme solution with BRISC at 2 nM in assay buffer was made by adding 10 μl of BRISC working stock (1 μM) to 5 ml of assay buffer. A 5 ml of substrate solution was made with 0.9 M of K63-diUbiquitin (made in-house) plus 0.1 M of IQF K63-diUbiquitin by adding the appropriate volumes of diUbiquitins to 5 ml of assay buffer. 45 μL of BRISC (enzyme solution) was dispensed into columns 1-11 of a 96 well PCR plate (skirted). Buffer was only dispensed in column 12 (no enzyme control). 45 μl of substrate solution was dispensed into columns 1-12 of a 96 well PCR plate (skirted). The plate was spun down at 3000 rpm (bench top centrifuge) at room temp for 2 min to remove any air bubbles. Using a Biomek FX, liquid handler (Bekman), 10 μl of BRISC (enzyme solution) was dispensed into the 4 quadrants of a 384 well plate. 200 nl of compounds was dispensed into each well of the 384 plates using the pin tool (columns A and B usually contain DMSO only, and serve as controls), and the plates were incubated for 10-15 minutes at room temperature. 10 μl of substrate solution was dispensed into the 4 quadrants of the 384 well plate. The plate was then incubated for 30 min at room temperature in the dark and read with settings suitable for detecting TAMRA fluorescence (excitation wavelength of 540 nm and emission wavelength of 580 nm). A PHERAStar plate (BMG labtech) reader was used with a gain value of 570. The Intensity/Background (I/Bg), defined as [(Average of columns 1 and 2; DMSO controls)/(Average of columns 23 and 24; no enzyme controls)], were about 5.

### Results and Discussion

The screen demonstrated an excellent Z′ factor of ~0.75. Several small molecule libraries totaling to -80,000 compounds were screened for the inhibition of BRISC activity. This resulted in the identification of seven distinct chemotypes and numerous singletons with activity in the range of 1-20 M. In particular, 51 compounds were defined as hits with a ~0.5% hit rate.

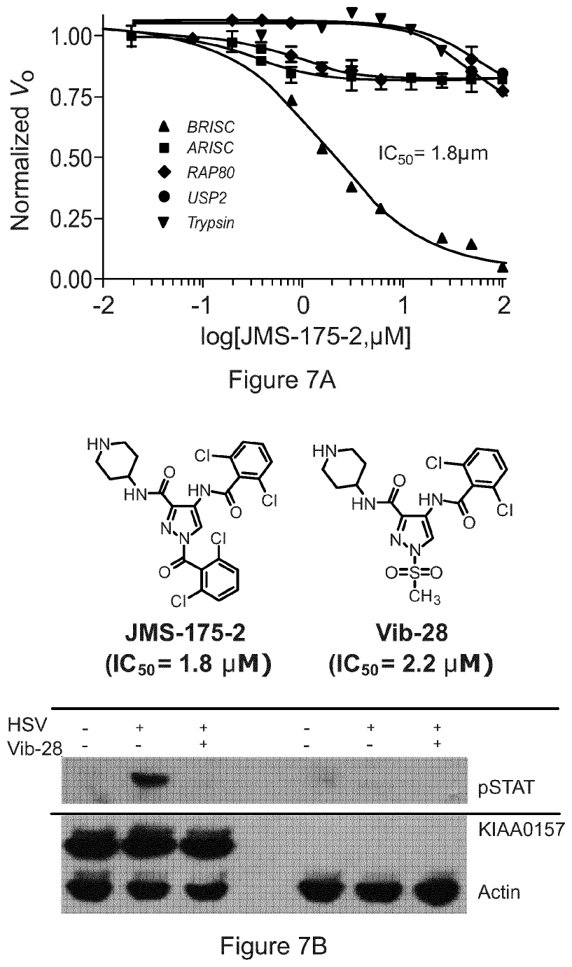

After identifying hit molecule, Compound 1, the structure of Compound 1 was confirmed and activity was determined following re-synthesis (FIGS. 7A,B). In particular, Compound 1 demonstrated selectivity for BRISC versus other DUBs (USP2) and serine proteases (trypsin) (FIG. 6C). Compound 1 showed no significant cross reactivity to the highly similar ARISC and RAP80 holoenzymes involved in DNA repair that shares 3 of 4 common subunits, including the BRCC36 catalytic subunit (FIG. 7A).

Compound 1 was modified to generate about 60 related analogs to provide an initial structure activity relationship (SAR). During this study, it was found that the small methane sulfonyl group replacement for the 2,6-di-chlorophenyl group resulted in an analog (Compound 2) that is equipotent to Compound 1, with a significantly lower molecular weight and improved drug-like properties (FIG. 7B, top). Importantly, the sulfonamide series is active in cells at 1-5 M, showing reduction in phosphorylated Stat1 following infection with a modified form of Herpes Simplex Virus that elicits robust interferon responses. For example, Compound 2 displayed remarkable efficacy at reducing interferon signaling in response to LPS in human cells at 5 μM in cells with no detectable cellular toxicity (FIG. 7B).

It was found that Compound 1 and Compound 2 are non-competitive inhibitors as increasing concentration of di-Ubiquitin substrate and free Zn²⁺ did not affect their ability to inhibit BRCC36 enzyme activity (See Table 1). The involvement of the MPN-domain (KIAA0157 instead of Abraxas) in determining the selectivity of Compound 1 also suggested that the compounds recognize an allosteric pocket formed by the higher order complex and not BRCC36 alone.

TABLE 1
Activity Range Table

| Compound # | BRISC IC₅₀ μM Range |
|---|---|
| 1 | 1-10 |
| 2 | 1-10 |
| 8 | 1-10 |
| 13 | 1-10 |
| 12 | 1-10 |
| 9 | 1-10 |
| 10 | 1-10 |
| 37 | 1-10 |
| 11 | 1-10 |
| 7 | 1-10 |
| 3 | 1-10 |
| 6 | 1-10 |
| 4 | 1-10 |
| 30 | 1-10 |
| 31 | 1-10 |
| 17 | 10-50 |
| 22 | 10-50 |
| 20 | 10-50 |
| 23 | 10-50 |
| 29 | 10-50 |
| 32 | 10-50 |
| 15 | 10-50 |
| 5 | 10-50 |
| 18 | 10-50 |
| 19 | 10-50 |
| 35 | 50-300 |
| 21 | 50-300 |
| 36 | 50-300 |
| 25 | 50-300 |
| 27 | 50-300 |
| 26 | 50-300 |
| 33 | 50-300 |
| 24 | 50-300 |
| 34 | 50-300 |

### Example 3: Crystal Structure of BRISC

BRCC36 lacks DUB activity when expressed as a single protein, but gains robust K63-Ub specific DUB activity in vitro upon interaction with KIAA0157. To understand the basis for regulated DUB activity, the X-ray crystal structure of the active BRISC complex was resolved at 2.5 angstroms resolution. The structure reveals extensive interactions between the Zn²⁺ active site containing MPN domain of BRCC36 and the MPN-domain of KIAA0157, and explains the molecular basis for regulation of DUB activity. The crystal structure can serve as a tool for the drug design approaches and for future co-crystallization studies.

### Example 4: Synthesis of Disclosed Compounds

This Example describes suitable synthesis schemes for making compounds according to the present disclosure. For example, Compound 1 can be formed according to Scheme 1 below with a core compound, I, as an intermediate:

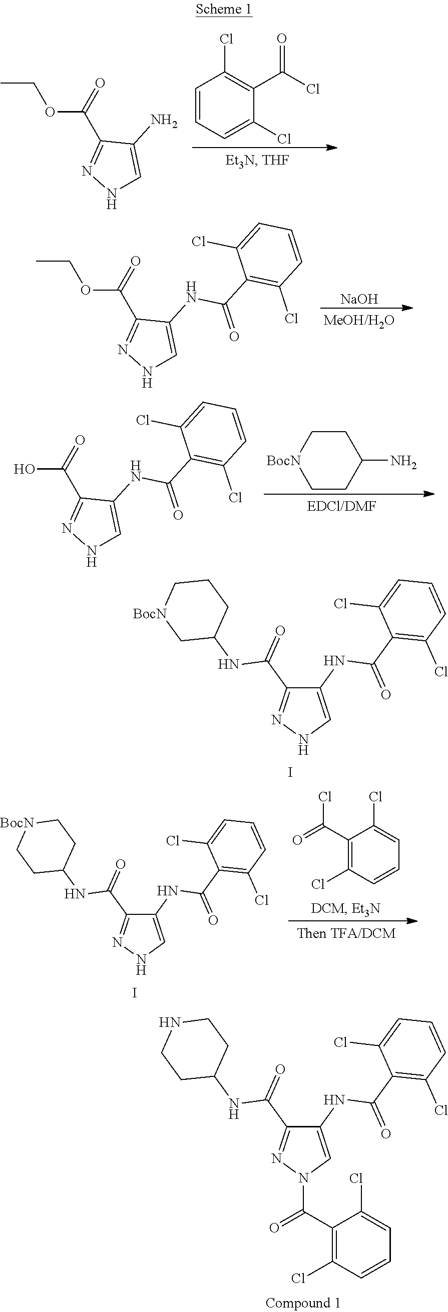

### Synthesis of 4-(2,6-dichlorobenzamido)-1-(2,6-dichlorobenzoyl)-N-(piperidin-4-yl)-1H-pyrazole-3-carboxamide, Compound 1

The following procedure is an example method used to synthesize Compound 1. The synthesis of tert-butyl 4-(4-(2,6-dichlorobenzamido)-1H-pyrazole-3-carboxamido)piperidine-1-carboxylate, I, was performed using published procedure (see WO 2007/129066; pg 73). 2,6-dichlorobenzolyl chloride (47.7 mg; 0.228 mmol) was added to a solution of I dissolved in dichloromethane (DCM) (10 mL) containing triethylamine (31.42 mg; 0.311 mmol) at 0° C., then allowed to warm to room temperature. The reaction was monitored by TLC until I was consumed (~1 h). The reaction mixture was then poured into 50 mL of ethyl acetate and washed with water, saturated sodium bicarbonate, water, 10% aq HCl solution, water, then brine. The ethyl acetate layer was dried over sodium sulfate, filtered, and concentrated on a rotoevaporator. The resulting residue was purified by flash chromatography (50% ethyl acetate in hexane) to yield tert-butyl 4-(4-(2,6-dichlorobenzamido)-1-(2,6-dichlorobenzoyl)-1H-pyrazole-3-carboxamido)piperidine-1-carboxylate (90.9 mg; 0.138 mmol; 67%). This was then treated with 20% TFA in DCM (2 mL) for 2 h, until the starting material disappeared by TLC. The reaction mixture was then concentrated on the rotoevaporator to provide the TFA salt of Compound 1 (92.35 mg; 100%). The ¹H NMR was concordant with structure. [M+1]+=556.

Scheme 1 can be used to synthesize compounds according to the disclosed subject matter, including, but not limited to Compounds 1, 7-13, 15, 17, 20, 22, 23, 28, 29, and/or 37, by further reaction or modification of Scheme 1, depending on the desired compound to be formed.

Moreover, the core compound, I, can be reacted according to Scheme 2 below to form sulfonamide analogs, such as Compound 2:

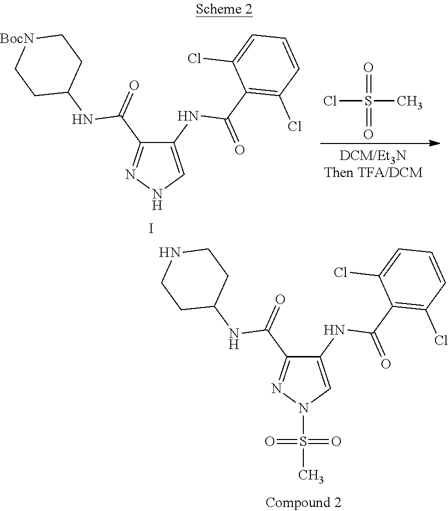

### Synthesis of 4-(2,6-dichlorobenzamido)-1-(methylsulfonyl)-N-(piperidin-4-yl)-1H-pyrazole-3-carboxamide, Compound 2

For purpose of example, Compound 2 can be synthesized according to the following procedure. The synthesis of tert-butyl 4-(4-(2,6-dichlorobenzamido)-1H-pyrazole-3-carboxamido) piperidine-1-carboxylate, I, was performed using the published procedure (see WO 2007/129066; pg 73). Methane sulfonyl chloride (30.3 mg; 0.265 mmol) was added to a solution of I (100 mg; 0.207 mmol) in DCM (10 mL) containing triethylamine (31.42 mg; 0.311 mmol) at 0° C. and allowed to warm to room temperature for 2 h until the starting material disappeared by TLC (60% ethyl acetate in hexane). The reaction mixture was then poured into 50 mL of ethyl acetate and washed with water, saturated sodium bicarbonate, water, 10% aq HCl solution, water, then brine. The ethyl acetate layer was dried over sodium sulfate, filtered, and concentrated on a rotoevaporator. The resulting residue was purified by flash chromatography (50% ethyl acetate in hexane) to yield tert-butyl 4-(4-(2,6-dichlorobenzamido)-1-(methylsulfonyl)-1H-pyrazole-3-carboxamido)piperidine-1-carboxylate (100.1 mg; 0.180 mmol; 87%). This was then treated with 20% TFA in DCM (2 mL) for 2 h, until starting material disappeared by TLC. The reaction mixture was then concentrated on the rotoevaporator to provide the TFA salt of Compound 2 (103.38 mg; 100%). The ¹H NMR was concordant with structure. [M+1]+=461.

For example, Scheme 2 can be used to synthesize Compounds 2, 3, 4, 5, 6, 14, 24, 25, 26, 27, 30, 31, 32, 33, and/or 34, by further reaction or modification of Scheme 2, depending on the desired compound to be formed.

Alternatively, the core compound, I, can be reacted according to Scheme 3 below to form urea analogs, such as Compound 18:

### Scheme 3

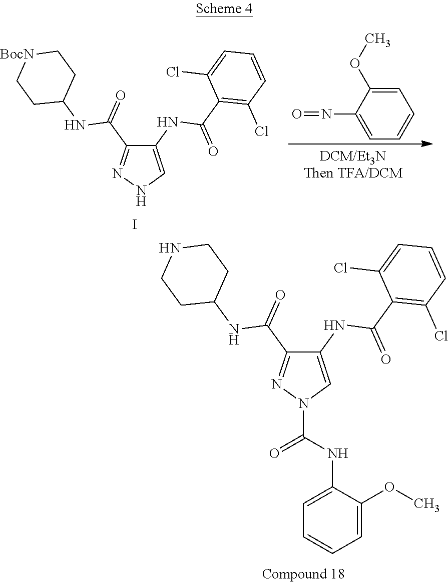

### Synthesis of 4-(2,6-dichlorobenzamido)-N1-(2-methoxyphenyl)-N3-(piperidin-4-yl)-1H-pyrazole-1,3-dicarboxamide, Compound 18

For purpose of example, Compound 18 can be synthesized according to the following procedure. Triethylamine (31.4 mg; 0.31 mmol) was added to a stirred solution of tert-butyl 4-(4-(2,6-dichlorobenzamido)-1H-pyrazole-3-carboxamido)piperidine-1-carboxylate, I, (100 mg, 17.79 mmol) in CH₂Cl₂ (5 mL), followed by addition of 1-isocyanato-2-methoxybenzene (37.1 mg; 0.25 mmol) drop wise at 0° C. The reaction mixture was allowed to warm to room temperature with stirring for 1 h until the starting material disappeared by TLC (60% ethyl acetate in hexane). The reaction mixture was then poured into 50 mL of ethyl acetate and washed with water, saturated sodium bicarbonate, water, 10% aq HCl solution, water, then brine. The ethyl acetate layer was dried over sodium sulfate, filtered, and concentrated on a rotoevaporator. The resulting residue was purified by flash chromatography (50% ethyl acetate in hexane) to yield tert-butyl 4-(4-(2,6-dichlorobenzamido)-1-((2-methoxyphenyl)carbamoyl)-1H-pyrazole-3-carboxamido)piperidine-1-carboxylate (105.9 mg; 0.17 mmol; 82%). This was then treated with 20% TFA in DCM (2 mL) for 2 h, until the starting material disappeared by TLC. The reaction mixture was then concentrated on the rotoevaporator to provide the TFA salt of Compound 18 (90.34 mg; 100%). The ¹H NMR was concordant with structure. [M+1]+=531.

For example, a slightly modified Scheme 3 can be used to synthesize Compounds 16, 18, 19, 21, 35, and/or 36.

Alternatively, Compounds 30, 32, 33, and 34 can be synthesized using the example procedure outlined below.

### Synthesis of 4-(2,6-dichlorobenzamido)-1-(4-(3-(1,3-dioxoisoindolin-2-yl)propoxy)phenylsulfonyl)-N-(piperidin-4-yl)-1H-pyrazole-3-carboxamide, Compound 30

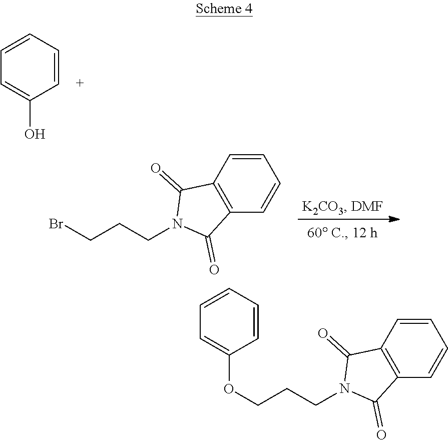

First, phenol and 2-(3-bromopropyl)isoindoline-1,3-dione were reacted according to Scheme 4. To a stirred solution of phenol (5 g, 53.12 mmol) in DMF (40 mL), potassium carbonate (14.68 g, 106.38 mmol) was added at room temperature, and stirred for 5 mins. 2-(3-bromopropyl)isoindoline-1,3-dione (14.2 g, 53.12 mmol) was added and the reaction mixture temperature was raised to 55° C. and the mixture was stirred for 18 h. Completion of the reaction was confirmed by TLC and the reaction mixture was filtered. The filtrate was concentrated under vacuum and the resultant residue was re-dissolved in ethyl acetate. The organic layer was washed with 5% aq NaOH and brine, dried over Na₂SO₄, and filtered. The filtrate was concentrated under vacuum to give 2-(3-phenoxypropyl)isoindoline-1,3-dione (11.2 g, 75%) as a white color solid, which was used for the next reaction without further purification.

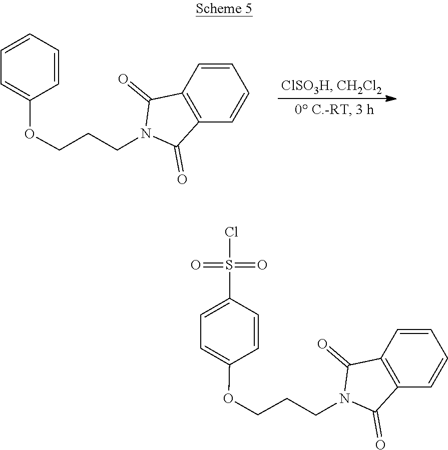 [SMILES: O=C1c2ccccc2C(=O)N1CCCOc1ccccc1 ; O=C1c2ccccc2C(=O)N1CCCOc1ccc(S(=O)(=O)Cl)cc1]

4-(3-(1,3-dioxoisoindolin-2-yl)propoxy)benzene-1-sulfonyl chloride was prepared from 2-(3-phenoxypropyl)isoindoline-1,3-dione according to Scheme 5. To a stirred solution of 2-(3-phenoxypropyl)isoindoline-1,3-dione (5.0 g, 17.79 mmol) in CH₂Cl₂ (30 mL), chlorosulphonic acid (2.34 ml, 35.58 mmol) was added drop wise at 0° C. The reaction mixture was brought to room temperature and stirred for 1 h. Completion of the reaction was monitored by TLC and then the reaction mixture was poured into an ice-water mixture (100 g). The product was extracted with a CH₂Cl₂ (2×50 mL) organic layer dried over anhydrous Na₂SO₄, filtered, and concentrated on a rotaevaporator. The resulting residue was purified by flash column chromatography (20% ethyl acetate and hexane) to yield 4-(3-(1,3-dioxoisoindolin-2-yl)propoxy)benzene-1-sulfonyl chloride (4.7 g, 12.40 mmol, 70%) as a white color solid. The ¹H NMR was concordant with structure.

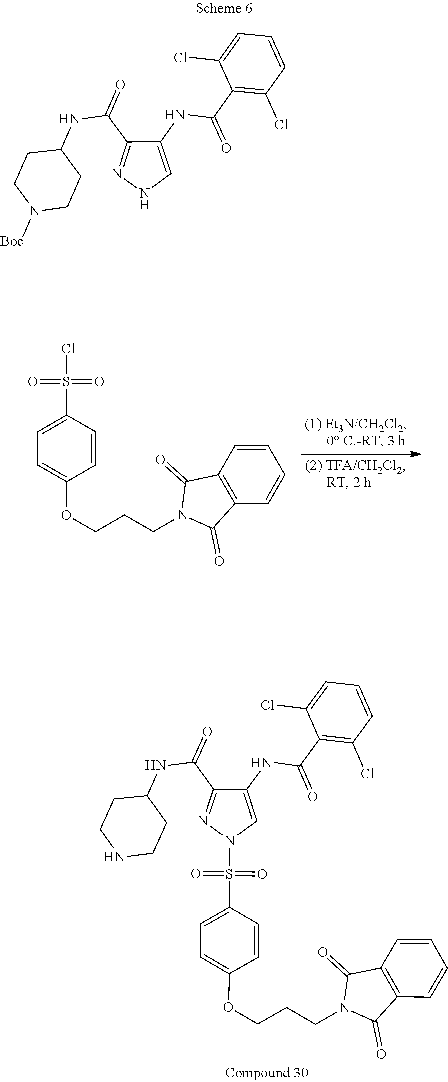 [SMILES: CC(C)(C)OC(=O)N1CCC(NC(=O)c2n[nH]cc2NC(=O)c2c(Cl)cccc2Cl)CC1 ; O=C1c2ccccc2C(=O)N1CCCOc1ccc(S(=O)(=O)Cl)cc1 ; O=C(NC1CCNCC1)c1nn(S(=O)(=O)c2ccc(OCCCN3C(=O)c4ccccc4C3=O)cc2)cc1NC(=O)c1c(Cl)cccc1Cl]

Compound 30 was prepared from 4-(3-(1,3-dioxoisoindolin-2-yl)propoxy)benzene-1-sulfonyl chloride and core compound, I, according to Scheme 6. To a stirred solution of compound tert-butyl 4-(4-(2,6-dichlorobenzamido)-1H-pyrazole-3-carboxamido)piperidine-1-carboxylate, I, (2.0 g, 4.15 mmol) in CH2Cl2 (30 mL), diisopropylethylamine (3.7 ml, 20.75 mmol), and 4-(3-(1,3-dioxoisoindolin-2-yl)propoxy)benzene-1-sulfonyl chloride (2.05 g, 5.39 mmol) were added simultaneously at 0° C. Then the reaction mixture was slowly brought to room temperature and stirred for 12 h. After completion of the reaction was confirmed by TLC, the reaction mixture was poured into ice cold water. The product was extracted with CH₂Cl₂ (2×30 mL) and washed with 1N aq HCl (20 ml), 5% aq NaHCO₃ (20 mL) and brine solution (20 mL). The organic layer was dried over anhydrous Na₂SO₄, filtered, and concentrated on a rotaevaporator. The resulting residue was purified by silica gel flash column chromatography (50% ethyl acetate and hexane) to give tert-butyl 4-(4-(2,6-dichlorobenzamido)-1-(4-(3-(1,3-dioxoisoindolin-2-yl)propoxy)phenylsulfonyl)-1H-pyrazole-3-carboxamido)piperidine-1-carboxylate (2.10 g 2.90 mmol, 70%) as a white color solid. The ¹H NMR was concordant with structure. [M+1]⁺=677. This compound (80 mg, 0.097 mmol) was treated with 20% TFA in CH₂Cl₂ in (2 ml) for 2 h, until the starting material disappeared by TLC. The reaction mixture was concentrated on the rotaevaporator to provide TFA salt of 4-(2,6-dichlorobenzamido)-1-(4-(3-(1,3-dioxoisoindolin-2-yl)propoxy)phenylsulfonyl)-N-(piperidin-4-yl)-1H-pyrazole-3-carboxamide (66 mg, 0.09 mmol, 95%). The ¹H NMR was concordant with structure. [M+1]⁺=725.

### Synthesis of 1-(4-(3-aminopropoxy)phenylsulfonyl)-4-(2,6-dichlorobenzamido)-N-(piperidin-4-yl)-1H-pyrazole-3-carboxamide, Compound 34

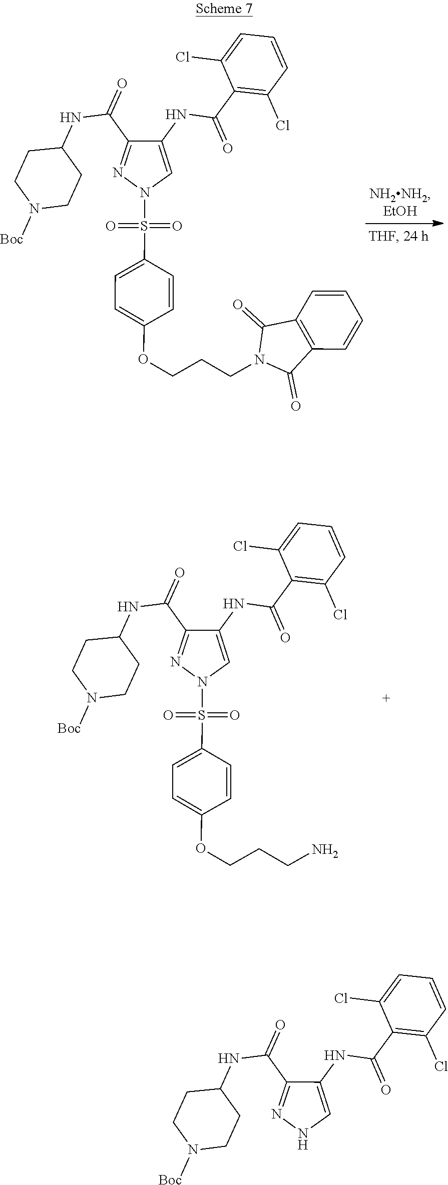 [SMILES: CC(C)(C)OC(=O)N1CCC(NC(=O)c2nn(S(=O)(=O)c3ccc(OCCCN4C(=O)c5ccccc5C4=O)cc3)cc2NC(=O)c2c(Cl)cccc2Cl)CC1 ; CC(C)(C)OC(=O)N1CCC(NC(=O)c2nn(S(=O)(=O)c3ccc(OCCCN)cc3)cc2NC(=O)c2c(Cl)cccc2Cl)CC1 ; CC(C)(C)OC(=O)N1CCC(NC(=O)c2n[nH]cc2NC(=O)c2c(Cl)cccc2Cl)CC1]

Tert-butyl 4-(1-(4-(3-aminopropoxy)phenylsulfonyl)-4-(2,6-dichlorobenzamido)-1H-pyrazole-3-carboxamido)piperidine-1-carboxylate was prepared from Compound 30 according to Scheme 7. To a stirred solution of tert-butyl 4-(4-(2,6-dichlorobenzamido)-1-(4-(3-(1,3-dioxoisoindolin-2-yl)propoxy)phenylsulfonyl)-1H-pyrazole-3-carboxamido)piperidine-1-carboxylate (300 mg, 0.363 mmol) in EtOH (10 mL), hydrazine monohydrate (0.36 mL, 7.26 mmol) was added at 0° C. and stirred for 16 h. The reaction mixture was filtered, and the residue was washed with 10 mL of EtOH. The combined filtrates were concentrated to a minimum volume by rotary evaporation. Resulting crude product was purified by flash column chromatography (10% MeOH in CH₂Cl₂) to give tert-butyl 4-(4-(2,6-dichlorobenzamido)-1H-pyrazole-3-carboxamido)piperidine-1-carboxylate (69 mg 0.14 mmol 40% biproduct) and tert-butyl 4-(1-(4-(3-aminopropoxy)phenylsulfonyl)-4-(2,6-dichlorobenzamido)-1H-pyrazole-3-carboxamido)piperidine-1-carboxylate (37 mg, 0.054 mmol, 15%) as a light-yellow, viscous oil. The ¹H NMR was concordant with structure. [M+1]⁺=695.

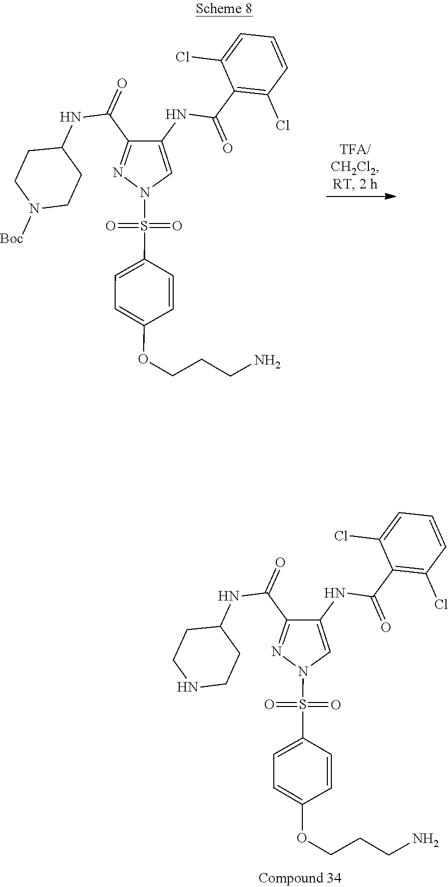 [SMILES: CC(C)(C)OC(=O)N1CCC(NC(=O)c2nn(S(=O)(=O)c3ccc(OCCCN)cc3)cc2NC(=O)c2c(Cl)cccc2Cl)CC1 ; NCCCOc1ccc(S(=O)(=O)n2cc(NC(=O)c3c(Cl)cccc3Cl)c(C(=O)NC3CCNCC3)n2)cc1]

Compound 34 was prepared from tert-butyl 4-(1-(4-(3-aminopropoxy)phenylsulfonyl)-4-(2,6-dichlorobenzamido)-1H-pyrazole-3-carboxamido)piperidine-1-carboxylate according to Scheme 8. Tert-butyl 4-(1-(4-(3-aminopropoxy)phenylsulfonyl)-4-(2,6-dichlorobenzamido)-1H-pyrazole-3-carboxamido)piperidine-1-carboxylate (30 mg, 0.043 mmol) was treated with 20% TFA in CH₂Cl₂ in (2 ml) for 2 h, until the starting material disappeared by TLC. The reaction mixture was concentrated on the rotaevaporator to provide TFA salt of 1-(4-(3-aminopropoxy)phenylsulfonyl)-4-(2,6-dichlorobenzamido)-N-(piperidin-4-yl)-1H-pyrazole-3-carboxamide (24 mg, 0.41 mmol, 95%). The ¹H NMR was concordant with structure. [M+1]⁺=595.

### Synthesis of Methyl 2-(4-(4-(2,6-dichlorobenzamido)-3-(piperidin-4-ylcarbamoyl)-1H-pyrazol-1-ylsulfonyl)phenoxy)acetate, Compound 33

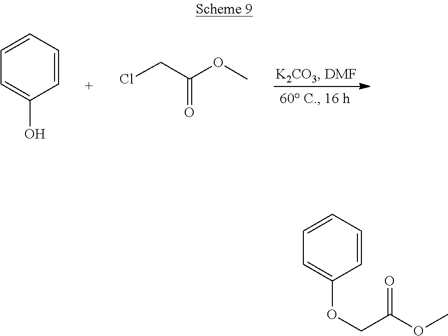

First, phenol and methyl chloroacetate were reacted according to Scheme 9 to form methyl 2-phenoxyacetate. To a stirred solution of phenol (5.0 g, 53.35 mmol) in DMF K₂CO₃ (14.75 g, 106.75 mmol), methyl chloroacetate (5.8 g, 53.35 mmol) was added. The reaction mixture was stirred at room temperature for 24 h. Completion of the reaction was confirmed by TLC and filtered. The filtrate was concentrated under vacuum and the resultant residue was re-dissolved in ethyl acetate. The organic layer was washed with NaOH and brine, dried over Na₂SO₄ and filtered. The filtrate was concentrated under vacuum to give methyl 2-phenoxyacetate (7.09 g, 80%) as a colorless oil, which was used for the next reaction without further purification.

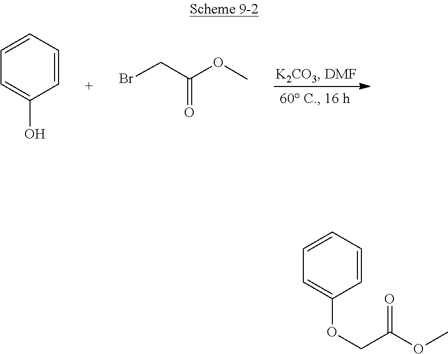

Alternatively, methyl 2-phenoxyacetate can be formed in accordance with Scheme 9-2. To a stirred solution of phenol (5.0 g, 53.35 mmol) in DMF K₂CO₃ (14.75 g, 106.75 mmol) was added and methyl bromoacetate (5.8 g, 53.35 mmol) respectively. The reaction mixture was stirred at room temperature for 24 h. Completion of the reaction confirmed by TLC and filtered. The filtrate was concentrated under vacuum and the resultant residue was redissolved in ethyl acetate. The organic layers were washed with NaOH and brine, dried over Na₂SO₄ and filtered. The filtrate was concentrated under vacuum to give methyl 2-phenoxyacetate (7.09 g, 80%) as a color less oil, which was used for the next reaction without further purification.

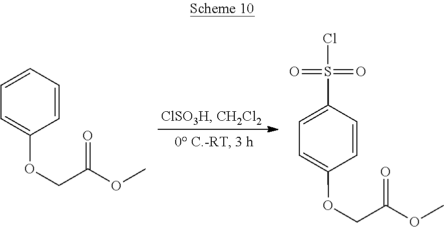 [SMILES: COC(=O)COc1ccccc1 ; COC(=O)COc1ccc(S(=O)(=O)Cl)cc1]

Methyl 2-(4-(chlorosulfonyl)phenoxy)acetate was prepared from methyl 2-phenoxyacetate according to Scheme 10. To a stirred solution of methyl 2-phenoxyacetate (5.0 g, 30.08 mmol) in CH₂Cl₂ (30 mL), chlorosulphonic acid (4.0 ml, 60.16 mmol) was added dropwise at 0° C. The reaction mixture was brought to room temperature and stirred for 1 h. After completion of the reaction, which was monitored by TLC (EtOAc/Hexanes=1/10), the reaction mixture was poured into an ice-water mixture (100 g). Product was extracted with CH₂Cl₂ (2×50 mL). The organic layer was dried over anhydrous Na₂SO₄, filtered, and concentrated on a rotaevaporator. The resulting residue was purified by flash column chromatography (20% ethyl acetate and hexane) to yield methyl 2-(4-(chlorosulfonyl)phenoxy)acetate (4.77 g, 18.05 mmol) as a white color solid. The ¹H NMR was concordant with structure.

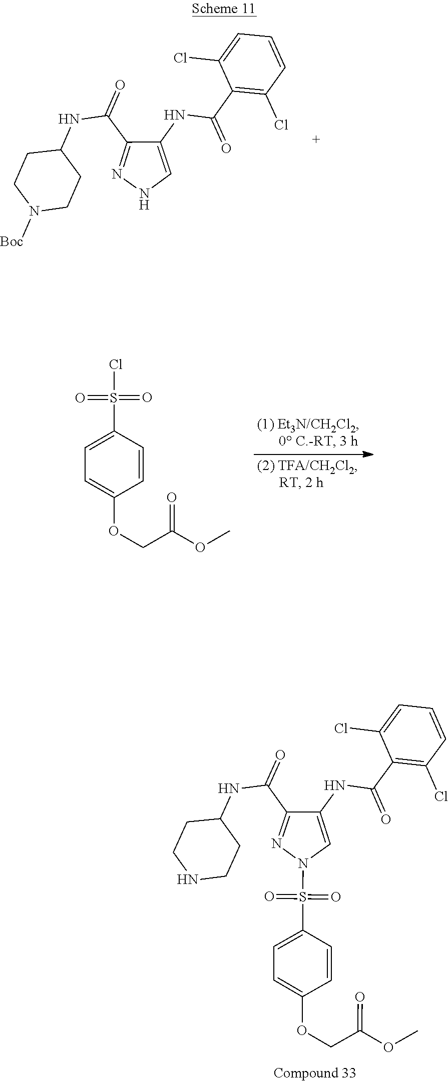 [SMILES: CC(C)(C)OC(=O)N1CCC(NC(=O)c2n[nH]cc2NC(=O)c2c(Cl)cccc2Cl)CC1 ; COC(=O)COc1ccc(S(=O)(=O)Cl)cc1 ; COC(=O)COc1ccc(S(=O)(=O)n2cc(NC(=O)c3c(Cl)cccc3Cl)c(C(=O)NC3CCNCC3)n2)cc1]

Compound 33 was prepared from methyl 2-(4-(chlorosulfonyl)phenoxy)acetate according to Scheme 11. To a stirred solution of tert-butyl 4-(4-(2,6-dichlorobenzamido)-1H-pyrazole-3-carboxamido)piperidine-1-carboxylate, I, (2.0 g, 4.15 mmol) in CH₂Cl₂ (30 mL), diisopropylethylamine (3.7 ml, 20.75 mmol) and methyl 2-(4-(chlorosulfonyl)phenoxy)acetate (1.43 g, 5.39 mmol) were added simultaneously at 0° C. Then, the reaction mixture was brought slowly to room temperature and stirred for 12 h. After confirmation of the reaction by TLC, the reaction mixture was poured into ice cold water. Product was extracted with CH₂Cl₂ (2×30 mL) and washed with 1N aq HCl (20 ml), 5% aq NaHCO₃ (20 mL) and brine solution (20 ml). The organic layer was dried over anhydrous Na₂SO₄, filtered, and concentrated on a rotaevaporator. The resulting residue was purified by flash column chromatography (20% ethyl acetate and hexane) to yield tert-butyl 4-(4-(2,6-dichlorobenzamido)-1-(4-(2-methoxy-2-oxoethoxy)phenylsulfonyl)-1H-pyrazole-3-carboxamido)piperidine-1-carboxylate (2.06 g 2.90 mmol, 70%) as a white color solid. The ¹H NMR was concordant with structure. [M+1]⁺=710.

This compound (100 mg, 0.14 mmol) was treated with 20% TFA in CH₂Cl₂ in (2 ml) for 2 h, until the starting material disappeared by TLC. The reaction mixture was concentrated on the rotoevaporator to provide TFA salt of Compound 33 (81 mg, 0.13 mmol, 95%). The ¹H NMR was concordant with structure. [M+1]⁺=610.

### Synthesis of Hex-5-ynyl 2-(4-(4-(2,6-dichlorobenzamido)-3-(piperidin-4-ylcarbamoyl)-1H-pyrazol-1-ylsulfonyl)phenoxy)acetate, Compound 32

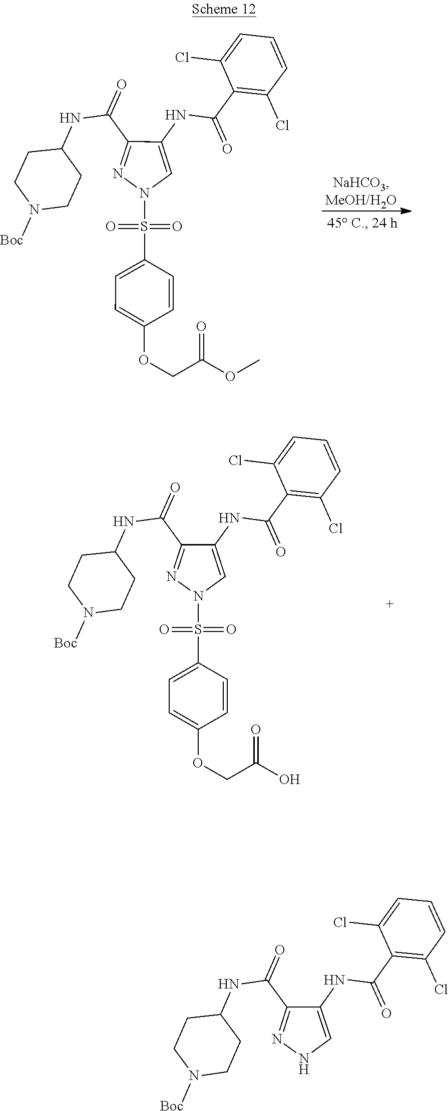 [SMILES: COC(=O)COc1ccc(S(=O)(=O)n2cc(NC(=O)c3c(Cl)cccc3Cl)c(C(=O)NC3CCN(C(=O)OC(C)(C)C)CC3)n2)cc1 ; CC(C)(C)OC(=O)N1CCC(NC(=O)c2nn(S(=O)(=O)c3ccc(OCC(=O)O)cc3)cc2NC(=O)c2c(Cl)cccc2Cl)CC1 ; CC(C)(C)OC(=O)N1CCC(NC(=O)c2n[nH]cc2NC(=O)c2c(Cl)cccc2Cl)CC1]

2-(4-(3-(1-(tert-butoxycarbonyl)piperidin-4-ylcarbamoyl)-4-(2,6-dichlorobenzamido)-1H-pyrazol-1-ylsulfonyl)phenoxy)acetic acid was prepared according to Scheme 12. To a stirred solution of tert-butyl 4-(4-(2,6-dichlorobenzamido)-1-(4-(2-methoxy-2-oxoethoxy)phenylsulfonyl)-1H-pyrazole-3-carboxamido)piperidine-1-carboxylate (1.1 g 1.55 mmol) in 50 mL (MeOH/H₂O 3:2), 130 mg of solid NaHCO₃ was added at room temperature. Then, the temperature was raised to 45° C. and stirred for 36 h. Completion of the reaction was confirmed by TLC. The reaction mixture solvent was evaporated under reduced pressure and the obtained reaction mixture crude was diluted with 30 mL water and acidified by 1N aq HCl pH 4. The product was extracted with CH₂Cl₂ (3×40 mL). The combined organic layer was dried over anhydrous sodium sulphate and filtered solvent was evaporated with a rotaevaporator. The resulting residue was purified by flash column chromatography (50% ethyl acetate and hexane) to yield tert-butyl 4-(4-(2,6-dichlorobenzamido)-1H-pyrazole-3-carboxamido)piperidine-1-carboxylate (660 mg, Bi product) and 4-(3-(1-(tert-butoxycarbonyl)piperidin-4-ylcarbamoyl)-4-(2,6-dichlorobenzamido)-1H-pyrazol-1-ylsulfonyl)phenoxy)acetic acid as a white solid (323 mg, 0477 mmol 30%). The ¹H NMR was concordant with structure. [M+1]⁺=696.

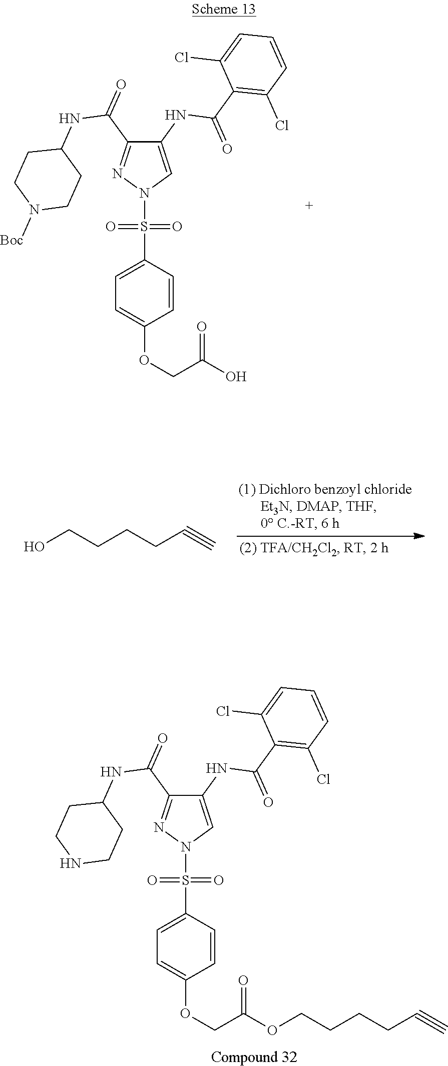

Compound 32 was prepared from 2-(4-(3-(1-(tert-butoxycarbonyl)piperidin-4-ylcarbamoyl)-4-(2,6-dichlorobenzamido)-1H-pyrazol-1-ylsulfonyl)phenoxy)acetic acid according to Scheme 13. To a stirred solution of 2-(4-(3-(1-(tert-butoxycarbonyl)piperidin-4-ylcarbamoyl)-4-(2,6-dichlorobenzamido)-1H-pyrazol-1-ylsulfonyl)phenoxy)acetic acid (100 mg, 0.31 mmol) in dry THF (10 mL), triethylamine (60 mg, 0.93 mmol), N,N-dimethyl amino pyridine (6 mg, 0.08 mmol), and dichlorobenzoyl chloride (30 mg, 0.31 mmol) were added at 0° C. and the reaction mixture was slowly brought to room temperature and stirred for 30 min. Then, the reaction mixture was cooled to 0° C. and 5-hexyn-1-ol (30 mg, 0.46 mmol) was added slowly and the reaction mixture was brought to room temperature and stirred for 18 h. Completion of the reaction was confirmed by TLC and the reaction mixture was cooled to 0° C. and quenched with cold water (10 mL). Product was extracted with an ethyl acetate (3×30 mL) organic layer and washed with 1N aq HCl (10 mL), saturated aq NaHCO₃ (10 mL), and brine solution (10 mL). The ethyl acetate layer was dried over anhydrous Na₂SO₄, filtered, and concentrated on a rotaevaporator. Resulting residue was purified by flash column chromatography (50% ethyl acetate and hexane) to yield tert-butyl 4-(4-(2,6-dichlorobenzamido)-1-(4-(2-(hex-5-ynyloxy)-2-oxoethoxy)phenylsulfonyl)-1H-pyrazole-3-carboxamido)piperidine-1-carboxylate (78 mg 0.10 mmol 70%). This was treated with 20% TFA in CH₂Cl₂ in (2 ml) for 2 h, until the starting material disappeared by TLC. The reaction mixture was concentrated on the rotaevaporator to provide TFA salt of Compound 32 (64 mg, 0.95 mmol, 95%). The ¹H NMR was concordant with structure. [M+1]⁺=676.

### Synthesis of 4-(2,6-Dichlorobenzamido)-1-(4-(hex-5-ynyloxy)phenylsulfonyl)-N-(piperidin-4-yl)-1H-pyrazole-3-carboxamide, Compound 41

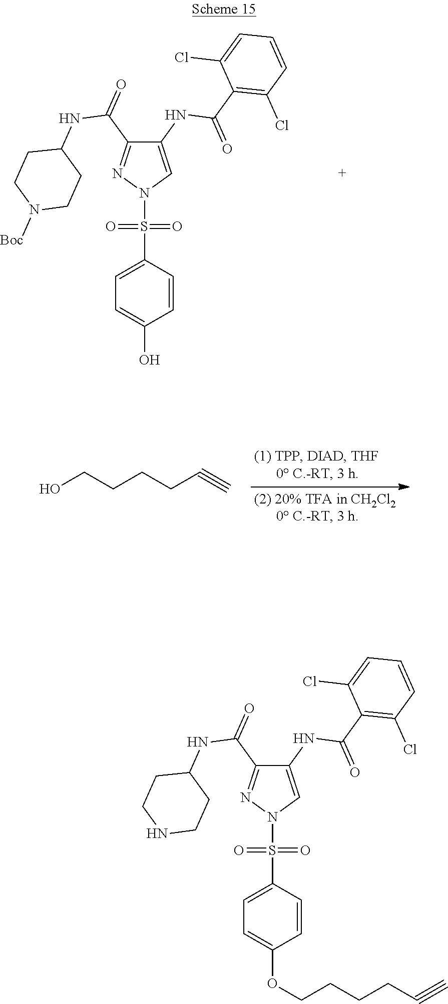

Compound 41 was prepared in accordance with Scheme 15. To a solution of 5-hexyn-1-ol 11 (0.105 g, 1.076 mmol) in THF (7 mL) at 0° C., containing Ph₃P (0.183 g, 0.699 mmol) and tert-butyl 4-(4-(2,6-dichlorobenzamido)-1-(4-hydroxyphenylsulfonyl)-1H-pyrazole-3-carboxamido)piperidine-1-carboxylate (0.343 g, 0538 mmol), was added DEAD (0.418 g, 0.699 mmol) dropwise at 0° C. for 3 mins and reaction mixture slowly brought to RT and stirred for 2 h. completion of the reaction confirmed by TLC, reaction mixture diluted with water product extracted with ethyl acetate and washed with brine solution dried anhydrous Na₂SO₄, solvent was removed under reduced pressure crude product purified by flash column afford (0.45 g yield 88%) product confirmed by ¹H NMR and LC-MS [M+1]⁺ 719.

### Synthesis of 4-(2,6-dichlorobenzamido)-1-(4-(4-(1-((S)-13-((2S,4S)-4-hydroxy-2-(4-(4-methylthiazol-5-yl)benzylcarbamoyl)pyrrolidine-1-carbonyl)-14,14-dimethyl-11-oxo-3,6,9-trioxa-12-azapentadecyl)-1H-1,2,3-triazol-4-yl)butoxy)phenylsulfonyl)-N-(piperidin-4-yl)-1H-pyrazole-3-carboxamide, Compound 43

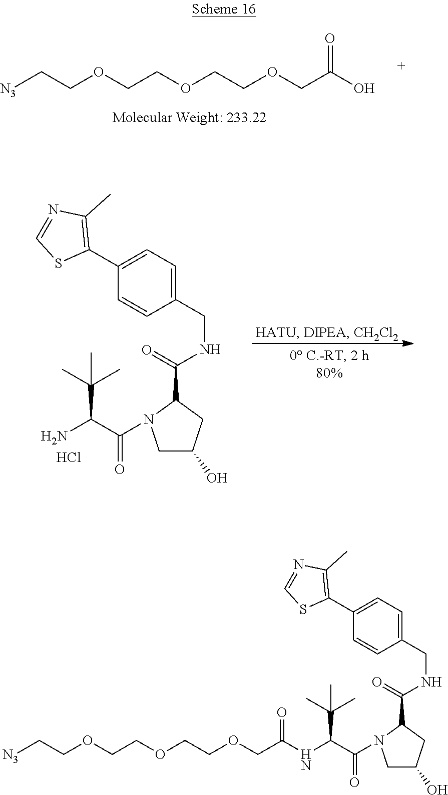

(2S,4S)-1-((S)-1-Azido-14,14-dimethyl-11-oxo-3,6,9-trioxa-12-azapentadecanecarbonyl)-4-hydroxy-N-(4-(4-methylthiazol-5-yl)benzyl)pyrrolidine-2-carboxamide was prepared in accordance with Scheme 16. To a solution of 2-(2-(2-(2-azidoethoxy)ethoxy)ethoxy)acetic acid (0.5 g 2.243 mmol, synthesized from reported procedure Journal of the American Chemical Society, 136(16), 5896-5899; 2014) and (2S,4S)-1-((S)-2-amino-3,3-dimethylbutanoyl)-4-hydroxy-N-(4-(4-methylthiazol-5-yl)benzyl)pyrrolidine-2-carboxamide hydrochloride salt ((1.002 g 2.243 mmol) in CH₂Cl₂ (15 ml) at 0° C. while stirring diisopropyl ethyl amine (0.830 g, 6.437 mmol) and HATU (0.978 g, 2.575 mmol) added simultaneously and reaction mixture brought to room temperature and stirring continued for 2 h completion of the reaction confirmed by TLC and quenched with cold water and reaction mixture diluted with cold water product extracted with CH₂Cl₂, dried over anhydrous Na₂SO₄, solvent removed under reduced pressure crude product purified by silica gel flash column afford (1.107 g, 80% yield) product confirmed by ¹H NMR and LC-MS LC-MS shows [M+1]⁺ 646.

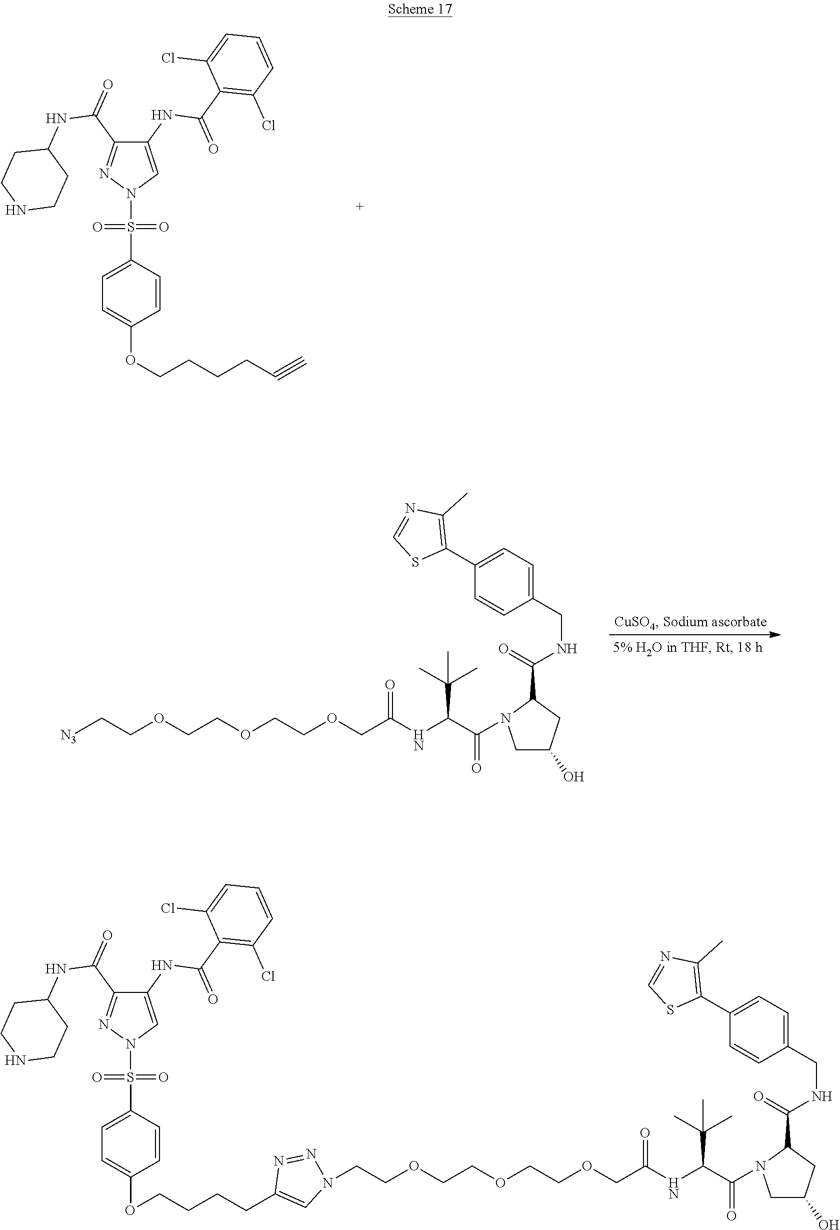

Compound 43 was prepared from (2S,4S)-1-((S)-1-Azido-14,14-dimethyl-11-oxo-3,6,9-trioxa-12-azapentadecanecarbonyl)-4-hydroxy-N-(4-(4-methylthiazol-5-yl)benzyl)pyrrolidine-2-carboxamide in accordance with Scheme 17. To a solution of 4-(2,6-dichlorobenzamido)-1-(4-(hex-5-ynyloxy)phenylsulfonyl)-N-(piperidin-4-yl)-1H-pyrazole-3-carboxamide (0.21 mg, 0.034 mmol) and (2S,4S)-1-((S)-1-azido-14,14-dimethyl-11-oxo-3,6,9-trioxa-12-azapentadecanecarbonyl)-4-hydroxy-N-(4-(4-methylthiazol-5-yl)benzyl)pyrrolidine-2-carboxamide (0.028 g, 0.034 mmol) in THF (2 ml) 5% Aqueous THF at 0° C., anhydrous copper sulphate (1.6 mg 0.0069 mmol) and sodium ascorbate (1.7 mg 0.0069) added simultaneously and stirred for 36 h at RT. Completion of the reaction confirmed by TLC, solvent evaporated product purified by reverse phase HPLC afford (15 mg 44% Yield). The ¹H NMR concordant with structure, LC-MS shows [M+1]⁺⁺/2=633.

### Synthesis of 1-(4-(4-(1-(2-(2-(2-(2-(4-((4S,5R)-2-(4-Tert-butyl-2-ethoxyphenyl)-4,5-bis(4-chlorophenyl)-4,5-dimethyl-4,5-dihydro-1H-imidazole-1-carbonyl)piperazin-1-yl)acetamido)ethoxy)ethoxy)ethyl)-1H-1,2,3-triazol-4-yl)butoxy)phenylsulfonyl)-4-(2,6-dichlorobenzamido)-N-(piperidin-4-yl)-1H-pyrazole-3-carboxamide, Compound 42

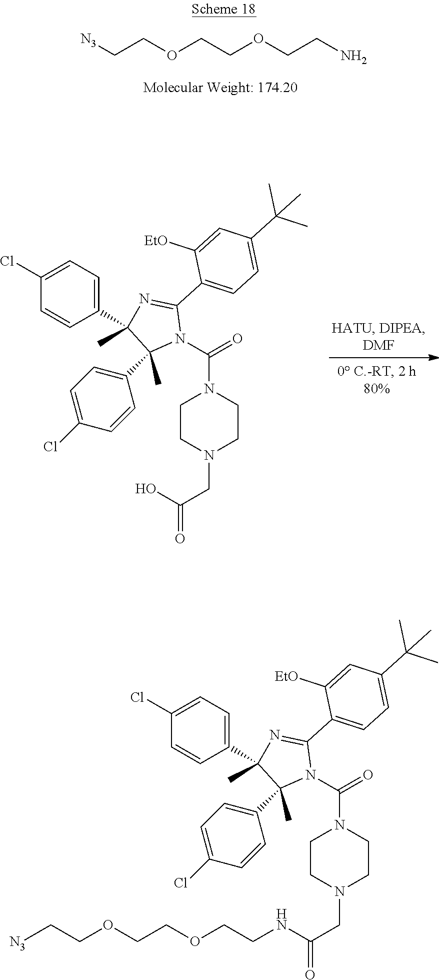 [SMILES: [N-]=[N+]=NCCOCCOCCN ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)O)CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCOCCOCCN=[N+]=[N-])CC1]

N-(2-(2-(2-azidoethoxy)ethoxy)ethyl)-2-(4-((4S,5R)-2-(4-tert-butyl-2-ethoxyphenyl)-4,5-bis(4-chlorophenyl)-4,5-dimethyl-4,5-dihydro-1H-imidazole-1-carbonyl)piperazin-1-yl)acetamide was prepared in accordance with Scheme 18. To a solution of 2-(4-((4S,5R)-2-(4-tert-butyl-2-ethoxyphenyl)-4,5-bis(4-chlorophenyl)-4,5-dimethyl-4,5-dihydro-1H-imidazole-1-carbonyl)piperazin-1-yl)acetic acid (0.6 g 0.915 mmol, 2-(2-(2-azidoethoxy)ethoxy)ethanamine (0.159 g 0.915 mmol) in DMF (5 ml) at 0° C. while stirring diisopropyl ethyl amine (048 ml, 2.745 mmol) and HATU (0.417 g, 1.098 mmol) added simultaneously and reaction mixture brought to rt and stirring continued for 2 h completion of the reaction confirmed by TLC and quenched with cold water and reaction mixture diluted with cold water product extracted with Ethyl acetate, dried over anhydrous Na₂SO₄, solvent removed under reduced pressure crude product purified by silica gel flash column afford (0.518, 70% yield) product confirmed by 1H NMR and LC-MS LC-MS shows [M+1]⁺=821.

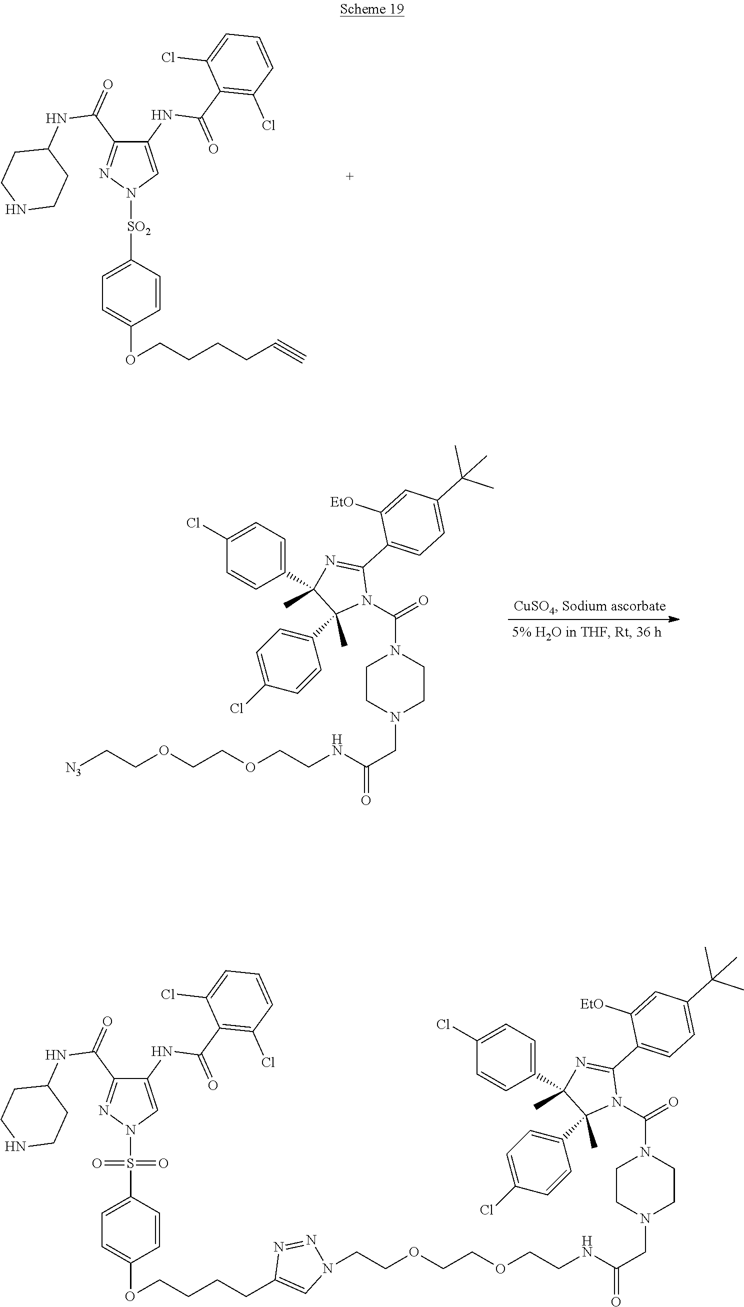 [SMILES: C#CCCCCOc1ccc(S(=O)(=O)n2cc(NC(=O)c3c(Cl)cccc3Cl)c(C(=O)NC3CCNCC3)n2)cc1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCOCCOCCN=[N+]=[N-])CC1 ; CCOc1cc(C(C)(C)C)ccc1C1=N[C@@](C)(c2ccc(Cl)cc2)[C@@](C)(c2ccc(Cl)cc2)N1C(=O)N1CCN(CC(=O)NCCOCCOCCn2cc(CCCCOc3ccc(S(=O)(=O)n4cc(NC(=O)c5c(Cl)cccc5Cl)c(C(=O)NC5CCNCC5)n4)cc3)nn2)CC1]

Compound 42 was prepared in accordance with Scheme 19. To a solution of 4-(2,6-dichlorobenzamido)-1-(4-(hex-5-ynyloxy)phenylsulfonyl)-N-(piperidin-4-yl)-1H-pyrazole-3-carboxamide (0.22 mg, 0.035 mmol) and N-(2-(2-(2-azidoethoxy)ethoxy)ethyl)-2-(4-((4S, 5R)-2-(4-tert-butyl-2-ethoxyphenyl)-4,5-bis(4-chlorophenyl)-4,5-dimethyl-4,5-dihydro-1H-imidazole-1-carbonyl)piperazin-1-yl)acetamide (0.03 g, 0.035 mmol) in THF (2 ml) 5% Aqueous THF at 0° C., anhydrous copper sulphate (1.6 mg 0.006 mmol) and sodium ascorbate (0 1.7 added simultaneously and stirred for 18 h at RT. Completion of the reaction confirmed by TLC, solvent evaporated product purified by reverse phase HPLC afford (16 mg 44% Yield). The ¹H NIR concordant with structure, LC-MS shows [M+1]⁺⁺/2=720.

### Example 5: Measuring the Activities of BRISC Inhibiting Compounds

The activities of the compounds disclosed in the present disclosure in inhibiting BRCC36 enzyme activity was measured in accordance with the method disclosed in Example 2. The results are shown in Table 2.

TABLE 2
Activity of BRISC inhibiting compounds.

| Compound # | BRISC IC₅₀ μM Range |
|---|---|
| 38 | 10-50 |
| 39 | 10-50 |
| 40 | 1-10 |
| 41 | 1-10 |
| 42 | 0.1-1 |
| 43 | 0.1-1 |
| 44 | 10-50 |
| 45 | 1-10 |
| 46 | 1-10 |
| 47 | 1-10 |
| 48 | 10-50 |
| 49 | 10-50 |
| 50 | 10-50 |
| 51 | 10-50 |
| 52 | 10-50 |
| 53 | 1-10 |
| 54 | 10-50 |
| 55 | 1-10 |
| 56 | 10-50 |
| 57 | 1-10 |
| 58 | 10-50 |
| 59 | 1-10 |
| 60 | 10-50 |
| 61 | 1-10 |
| 62 | 1-10 |
| 63 | 10-50 |
| 64 | 10-50 |

### REFERENCES

1. Haglund, K. & Dikic, I. Ubiquitylation and cell signaling. Embo J 24, 3353-9 (2005).
2. Terzic, J., Marinovic-Terzic, I., Ikeda, F. & Dikic, I. Ubiquitin signals in the NF-kappaB pathway. Biochemical Society transactions 35, 942-5 (2007).
3. Cooper, E. M. et al. K63-specific deubiquitination by two JAMM/MPN+ complexes: BRISC-associated Brcc36 and proteasomal Pohl. Embo J 28, 621-31 (2009).
4. Yao, T. & Cohen, R. E. A cryptic protease couples deubiquitination and degradation by the proteasome. Nature 419, 403-7 (2002).
5. Verma, R. et al. Role of Rpn11 metalloprotease in deubiquitination and degradation by the 26S proteasome. Science 298, 611-5 (2002).
6. Zheng, H. et al. A BRISC-SHMT complex deubiquitinates IFNAR1 and regulates interferon responses. Cell Rep 5, 180-93 (2013).
7. Zeqiraj, E. et al. Higher-Order Assembly of BRCC36-KIAA0157 Is Required for DUB Activity and Biological Function. Mol Cell 59, 970-83 (2015).
8. Baechler, E. C. et al. Interferon-inducible gene expression signature in peripheral blood cells of patients with severe lupus. Proc Natl Acad Sci USA 100, 2610-5 (2003).
9. Bennett, L. et al. Interferon and granulopoiesis signatures in systemic lupus erythematosus blood. J Exp Med 197, 711-23 (2003).
10. Nacionales, D. C. et al. Deficiency of the type I interferon receptor protects mice from experimental lupus. Arthritis Rheum 56, 3770-83 (2007).
11. Reeves, W. H., Lee, P. Y., Weinstein, J. S., Satoh, M. & Lu, L. Induction of autoimmunity by pristane and other naturally occurring hydrocarbons. Trends Immunol 30, 455-64 (2009).
12. Stewart, T. A. Neutralizing interferon alpha as a therapeutic approach to autoimmune diseases. Cytokine & growth factor reviews 14, 139-54 (2003).
13. McBride, J. M. et al. Safety and pharmacodynamics of rontalizumab in patients with systemic lupus erythematosus: results of a phase I, placebo-controlled, double-blind, dose-escalation study. Arthritis and rheumatism 64, 3666-76 (2012).
14. Satoh, M. & Reeves, W. H. Induction of lupus-associated autoantibodies in BALB/c mice by intraperitoneal injection of pristane. J Exp Med 180, 2341-6 (1994).
15. Urbonaviciute, V. et al. Toll-like receptor 2 is required for autoantibody production and development of renal disease in pristane-induced lupus. Arthritis Rheum 65, 1612-23 (2013).
16. Christensen, S. R. et al. Toll-like receptor 7 and TLR9 dictate autoantibody specificity and have opposing inflammatory and regulatory roles in a murine model of lupus. Immunity 25, 417-28 (2006).
17. Savarese, E. et al. Requirement of Toll-like receptor 7 for pristane-induced production of autoantibodies and development of murine lupus nephritis. Arthritis Rheum 58, 1107-15 (2008).
18. Liu, Z. & Davidson, A. Taming lupus—a new understanding of pathogenesis is leading to clinical advances. Nat Med 18, 871-82 (2012).
19. Nashi, E., Wang, Y. & Diamond, B. The role of B cells in lupus pathogenesis. Int J Biochem Cell Biol 42, 543-50 (2010).
20. Crow, M. K. Type I interferon in the pathogenesis of lupus. J Immunol 192, 5459-68 (2014).
21. Dorner, T., Giesecke, C. & Lipsky, P. E. Mechanisms of B cell autoimmunity in SLE. Arthritis Res Ther 13, 243 (2011).
22. Kirou, K. A., Salmon, J. E. & Crow, M. K. Soluble mediators as therapeutic targets in systemic lupus erythematosus: cytokines, immunoglobulin receptors, and the complement system. Rheum Dis Clin North Am 32, 103-19, ix (2006).
23. Py, B. F., Kim, M. S., Vakifahmetoglu-Norberg, H. & Yuan, J. Deubiquitination of NLRP3 by BRCC3 critically regulates inflammasome activity. Mol Cell 49, 331-8 (2013).
24. Patterson-Fortin, J., Shao, G., Bretscher, H., Messick, T. E. & Greenberg, R. A. Differential regulation of JAMM domain deubiquitinating enzyme activity within the RAP80 complex. J Biol Chem 285, 30971-81.
25. Lee, P. Y. et al. TLR7-dependent and FcgammaR-independent production of type I interferon in experimental mouse lupus. J Exp Med 205, 2995-3006 (2008).
26. Sims, J. J. et al. Polyubiquitin-sensor proteins reveal localization and linkage-type dependence of cellular ubiquitin signaling. Nat Methods 9, 303-9 (2012).
27. van Wijk, S. J. et al. Fluorescence-based sensors to monitor localization and functions of linear and k63-linked ubiquitin chains in cells. Mol Cell 47, 797-809 (2012).
28. Kessler, B. M. Ubiquitin—omics reveals novel networks and associations with human disease. Current opinion in chemical biology 17, 59-65 (2013).
29. Sobhian, B. et al. RAP80 targets BRCA1 to specific ubiquitin structures at DNA damage sites. Science 316, 1198-202 (2007).
30. Greenberg, R. A. et al. Multifactorial contributions to an acute DNA damage response by BRCA1/BARD1-containing complexes. Genes Dev 20, 34-46 (2006).
31. Shao, G. et al. MERIT40 controls BRCA1-Rap80 complex integrity and recruitment to DNA double-strand breaks. Genes Dev 23, 740-54 (2009).
32. Shao, G. et al. The Rap80-BRCC36 de-ubiquitinating enzyme complex antagonizes RNF8-Ubc13-dependent ubiquitination events at DNA double strand breaks. Proc Natl Acad Sci USA 106, 3166-71 (2009).
33. Sakamoto, K. M. et al. Protacs: chimeric molecules that target proteins to the Skp1-Cullin-F box complex for ubiquitination and degradation. Proc Natl Acad Sci USA 98, 8554-9 (2001).
34. Sakamoto, K. M. et al. Development of Protacs to target cancer-promoting proteins for ubiquitination and degradation. Mol Cell Proteomics 2, 1350-8 (2003).
35. Lu, J. et al. Hijacking the E3 Ubiquitin Ligase Cereblon to Efficiently Target BRD4. Chem Biol 22, 755-63 (2015).
36. Rodriguez-Gonzalez, A. et al. Targeting steroid hormone receptors for ubiquitination and degradation in breast and prostate cancer. Oncogene 27, 7201-11 (2008).

The contents of all figures and all references, patents and published patent applications and Accession numbers cited throughout this application are expressly incorporated herein by reference.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the systems and methods of the disclosed subject matter without departing from the spirit or scope of the disclosed subject matter. Thus, it is intended that the disclosed subject matter include modifications and variations that are within the scope of the appended claims and their equivalents.

1. A compound of Formula I: 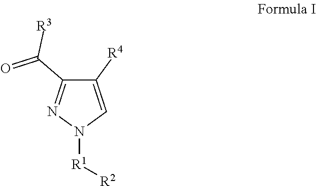
or a salt, ester, or solvate thereof,
wherein R¹ is C═O, SO₂-Me, carbonyl, or a sulfonyl;
wherein R² is optional, and if present, is CF₃, C₁₋₆alkyl, substituted C₁₋₆alkyl, amino, alkoxycarbonyl, aryl, substituted aryl, benzyl, or a substituted naphthalene;
wherein R³ is a substituted amino; and
wherein R⁴ is H or an amino.

2. The compound of claim 1, wherein
R¹ is C═O, SO₂-Me, carbonyl, or a sulfonyl;
R² is optional, and if present, is CF₃, C₁₋₆alkyl, substituted C₁₋₆alkyl, amino, alkoxycarbonyl, aryl, substituted aryl, benzyl, or a substituted naphthalene, wherein
the C₁₋₆alkyl is substituted with phenyl substituted with —OC₁₋₆alkyl,
the aryl group is substituted with one, two or three groups selected from: halo, —OR⁷, haloalkyl, —NH₂, C₁₋₆alkylamino (such as methylamine or dimethylamine), and C₁₋₆alkyl ester;
the naphthalene is substituted with a C₁₋₆alkylamino;
the amino is selected from: —NHR⁸;
R³ is a substituted amino, wherein the amino is substituted with two groups (optionally where 1 group is H or Me) selected from: H, C₁₋₆alkyl, —(CH₂)ₘNH₂, substituted or unsubstituted 6 membered heteroaryl, substituted or unsubstituted —(CH₂)ₘ-4 to -6-membered heterocycloalkyl or the two substituents on the amino may form a substituted or unsubstituted 5 or 6 membered ring,
wherein each group that may be substituted is substituted with one or two C₁₋₆alkyl groups;
R⁴ is H or a substituted amino, wherein the amino group is substituted with benzyl, —C(O)aryl or —C(O)heteroaryl wherein the benzyl group and the aryl group are unsubstituted or substituted with R¹⁰; and
R⁷ is selected from: C₁₋₆alkyl, C₁₋₆alkynyl, —CH₂C(O)OH, —CH₂C(O)O(CH₂)ₙCH₃, —CH₂C(O)O(CH₂)ₙCCH, —CH₂C(O)NH(CH₂)ₙCH₃, —(CH₂)ₙNH₂, —(CH₂)ₙNO₂, and —(CH₂)ₙR⁹,
R⁸ is phenyl substituted with halo, —CO₂H, —CO₂—C₁₋₆alkyl, and —OC₁₋₆alkyl;
R⁹ is phthalimide;
R¹⁰ is selected from: C₁₋₆alkyl, —OC₁₋₆alkyl, C₁₋₆haloalkyl, halo, —NO₂, —CH₂morpholinyl and diaziridine substituted with C₁₋₆haloalkyl; and
n is 0 to 6
m is 0 to 4.

3. The compound of claim 1, wherein the compound is a compound of Formula II: 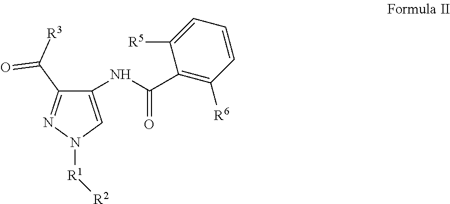
or a salt, ester, or solvate thereof,
wherein R¹ is C═O, SO₂-Me, carbonyl, or a sulfonyl;
wherein R² is optional, and if present, is CF₃, C₁₋₆alkyl, substituted C₁₋₆alkyl, amino, alkoxycarbonyl, aryl, substituted aryl, benzyl, or a substituted naphthalene;
wherein R³ is a substituted amino; and
wherein R⁵ and R⁶ are independently H or a halo.

4. The compound of claim 1, wherein the compound is a compound of Formula III 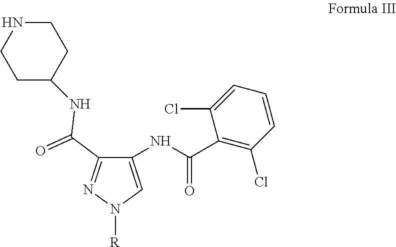
wherein R is SO₂-Me, benzoyl, substituted benzoyl, or a sulfonyl.

5. The compound of claim 4, wherein R is a CO-2,6-dichlorobenzoyl.

6. The compound of claim 4, wherein R is a SO₂-Me.

7. The compound of claim 1, wherein the compound is

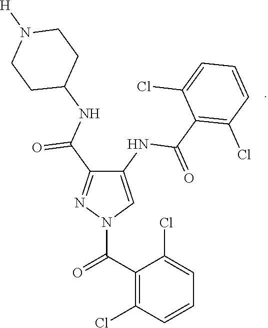 [SMILES: O=C(NC1CCNCC1)c1nn(C(=O)c2c(Cl)cccc2Cl)cc1NC(=O)c1c(Cl)cccc1Cl]

8. The compound of claim 1, wherein the compound is

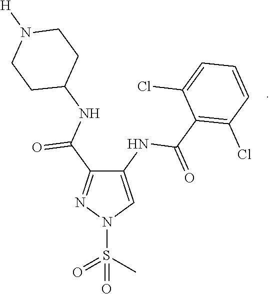 [SMILES: CS(=O)(=O)n1cc(NC(=O)c2c(Cl)cccc2Cl)c(C(=O)NC2CCNCC2)n1]

9. A pharmaceutical composition, comprising the compound of claim 1 and a pharmaceutically acceptable carrier.

10. A method for treating or preventing an autoimmune disease, the method comprising administering a therapeutically effective amount of the compound of claim 1 to a patient.

11. The method of claim 10, wherein the autoimmune disease is systemic lupus erythematosus, systemic sclerosis, scleroderma, myositis, or Sjögren's syndrome.

12. A method for treating or preventing an inflammatory disease, the method comprising administering a therapeutically effective amount of the compound of claim 1 to a patient.

13. A method, comprising administering a therapeutically effective amount of the compound of claim 1 to a patient.

14. A method, comprising administering a therapeutically effective amount of the compound of claim 2 to a patient.

15. A method, comprising administering a therapeutically effective amount of the compound of claim 3 to a patient.

16. A method, comprising administering a therapeutically effective amount of the compound of claim 4 to a patient.

17. A method, comprising administering a therapeutically effective amount of the compound of claim 5 to a patient.

18. A method, comprising administering a therapeutically effective amount of the compound of claim 6 to a patient.

19. A method, comprising administering a therapeutically effective amount of the compound of claim 7 to a patient.

20. A method, comprising administering a therapeutically effective amount of the compound of claim 8 to a patient.
